(19) United States
(12) Patent Application Publication
(10) Pub. No.: US 2019/0314072 A1
(43) Pub. Date: Oct. 17, 2019

(54) FIXTURE FOR FUNNEL CHEST CORRECTION BAR AND FUNNEL CHEST CORRECTION DEVICE

(71) Applicant: KAWASAKI GAKUEN EDUCATIONAL FOUNDATION, Kurashiki-shi (JP)

(72) Inventors: Sadashige Uemura, Okayama-shi (JP); Ryushi Takayama, Tokyo (JP)

(73) Assignee: KAWASAKI GAKUEN EDUCATIONAL FOUNDATION, Kurashiki-shi (JP)

(21) Appl. No.: 16/452,997

(22) Filed: Jun. 26, 2019

Related U.S. Application Data
(63) Continuation of application No. PCT/JP2017/046027, filed on Dec. 21, 2017.

(30) Foreign Application Priority Data
Jan. 5, 2017 (JP) 2017-000357

(51) Int. Cl. A61B 17/80; A61B 17/86

(52) U.S. Cl. CPC A61B 17/8076; A61B 2017/681; A61B 17/86; A61B 17/8023

(57) ABSTRACT

This fixture for securing a bar for funnel chest correction to a chest of a patient, includes: a base member having a length that allows the base member to extend across a plurality of ribs; a pressing member configured to be overlaid on the base member; a protrusion formed on an upper face of the base member; a plurality of screw holes formed in the base member; a plurality of holes formed in the pressing member at positions corresponding to the respective screw holes; and a plurality of screws. The pressing member is overlaid on the base member in a state where two bars are respectively disposed on both sides of the protrusion, and a screw is screwed into any of the screw holes on each of both sides of the protrusion, whereby the two bars are fixed by being sandwiched by the base member and the pressing member.

### BACKGROUND OF THE INVENTION

### 1. Field of the Invention

The present invention relates to a fixture for stably securing a bar for funnel chest correction to the chest of a patient and a funnel chest correction device using the fixture.

### 2. Disclosure of Related Art

As a method for correcting a funnel chest, the Nuss procedure has widely been known, for example. In this method, an arch-shaped stainless bar is inserted into the body, and then, the sternum is pushed up by the bar from inside the body, whereby funnel chest correction is performed. Specifically, incisions are made at both sides of a patient in order to pass the arch-shaped bar therethrough, and the bar is inserted into the thoracic cavity from the intercostal muscle between ribs. At this time, the arch-shaped bar is in a downwardly protruding state. Accordingly, a center portion of the bar is located inside the sternum of the patient. At this time, both ends of the bar are in a state of protruding from both sides of the patient. In this state, the bar is turned by 180 degrees to make the bar protrude upwardly, whereby the sternum is pushed up by the bar. Then, both ends of the bar are inserted under the skin, and both ends of the bar are bound to the ribs with a surgical suture. A single bar may be used, but two bars may be used when the concaveness of the chest is significant.

According to this method, when the sternum is pushed up by the bar being turned by 180 degrees, the bent ribs and the costal cartilage are corrected into normal shapes. Stabilization of the corrected shapes takes several weeks, during which the bar serves as a splint to help recovery from linear fracture. For this correction, the bar is left in the body of the patient for several years. Therefore, preferably, the bar is stably secured so as not to be dislocated or return to the original position from the state of being turned by 180 degrees. Conventionally, a measure of binding both ends of the bar to the ribs with a surgical suture has been taken. However, such a measure does not fully ensure the stability of the bar.

Recently, a method in which a fixture (stabilizer) for preventing rotation of the bar is attached to each end of the bar has also been employed.

For example, US Patent Application Publication No. 2010/0256691 describes a method in which each end portion of a bar is fixed by use of a fixture. In this method, the end portion of the bar is inserted in a groove of the fixture, and the fixture is placed to the outside of two ribs so as to extend across the two ribs. In this state, the fixture is bound to the two ribs, and accordingly, the end portion of the bar is secured.

US Patent Application Publication No. 2010/0256691 also describes a method in which each end portion of a bar is fixed by a pair of frame members each having a long hole. According to this method, the pair of frame members are overlaid at a position of a hole of the end portion of the bar. In this state, a bolt is passed through the long holes of the pair of frame members and the hole of the end portion of the bar. Then, a nut is fitted to the bolt. Further, the pair of frame members are hung over two ribs, respectively, and in this state, the bolt and the nut are fastened together. As a result, the pair of frame members are fixed to the end portion of the bar, whereby the end portion of the bar is secured.

Of the methods described in US Patent Application Publication No. 2010/0256691, in the case of the method in which each end portion of the bar is inserted into the groove of the fixture, the end portion of the bar and the fixture are not fixed to each other. Thus, the end portion of the bar is in an unstable state in which the end portion is movable along the groove. Therefore, the end portion of the bar could come off from the groove of the fixture. Therefore, this method is not sufficient for stably securing the bar to the chest of a patient.

Meanwhile, the method in which each end portion is secured by means of two frame members requires very troublesome operations during surgery, such as the operation of overlaying the two frame members on the end portion of the bar and passing the bolt through each hole, the operation of fitting the nut to the bolt, and further, the operation of hanging the two frame members over the ribs and fastening the bolt and the nut together.

### SUMMARY OF THE INVENTION

A first mode of the present invention relates to a fixture for securing a bar for funnel chest correction to the chest of a patient. The fixture according to this mode includes: a base member having a length that allows the base member to extend across a plurality of ribs; a pressing member configured to be overlaid on the base member; a protrusion formed on an upper face of the base member; a plurality of screw holes formed in the base member so as to be arranged side by side in a longitudinal direction on both sides of the protrusion of the base member; a plurality of holes formed in the pressing member at positions corresponding to the respective screw holes; and a plurality of screws. The pressing member is overlaid on the base member in a state where two bars are respectively disposed on both sides of the protrusion, and a screw is screwed into any of the screw holes on each of both sides of the protrusion, whereby the two bars are fixed by being sandwiched by the base member and the pressing member.

A second mode of the present invention relates to a fixture for securing a bar for funnel chest correction to the chest of a patient. The fixture according to this mode includes: a base member having a length that allows the base member to extend across a plurality of ribs; a groove portion that is formed in the base member and into which an end portion of the bar is slidably fitted; a screw hole formed in a face on the groove portion side of the base member; a screw configured to be screwed into the screw hole; and a pressing part configured to press the end portion of the bar fitted in the groove portion, against the groove portion through fastening of the screw onto the base member, thereby fixing the end portion to the groove portion.

A third mode of the present invention relates to a funnel chest correction device. The funnel chest correction device according to this mode includes: a fixture configured to secure a bar for funnel chest correction to a chest of a patient; and the bar for funnel chest correction of which end portion is to be fixed to the fixture. The fixture includes: a base member having a length that allows the base member to extend across a plurality of ribs; a pressing member configured to be overlaid on the base member; a protrusion formed on an upper face of the base member; a plurality of screw holes formed in the base member so as to be arranged side by side in a longitudinal direction on both sides of the protrusion of the base member; a plurality of holes formed in the pressing member at positions corresponding to the respective screw holes; and a plurality of screws. The pressing member is overlaid on the base member in a state where two bars are respectively disposed on both sides of the protrusion, and a screw is screwed into any of the screw holes on each of both sides of the protrusion, whereby the two bars are fixed by being sandwiched by the base member and the pressing member.

According to the funnel chest correction device of this this mode, effects similar to those in the first mode are exhibited.

A fourth mode of the present invention relates to a funnel chest correction device. The funnel chest correction device according to this mode includes: a fixture configured to secure a bar for funnel chest correction to a chest of a patient; and the bar for funnel chest correction of which end portion is to be fixed to the fixture. The fixture includes: a base member having a length that allows the base member to extend across a plurality of ribs; a groove portion that is formed in the base member and into which an end portion of the bar is slidably fitted; a screw hole formed in a face on the groove portion side of the base member; a screw configured to be screwed into the screw hole; and a pressing part configured to press the end portion of the bar fitted in the groove portion, against the groove portion through fastening of the screw onto the base member, thereby fixing the end portion to the groove portion.

According to the funnel chest correction device of this mode, effects similar to those in the second mode are exhibited.

### BRIEF DESCRIPTION OF THE DRAWINGS

The above and other objects and new features of the present invention will be fully clarified by the following description of the embodiment, when read in conjunction with accompanying drawings.

FIG. 1 is an exploded perspective view of a fixture according to Embodiment 1;

FIGS. 2A to 2C are a plan view, a side view, and a rear view, respectively, each showing a configuration of a base member according to Embodiment 1;

FIGS. 3A to 3C are a plan view, a side view, and a rear view, respectively, each showing a configuration of a first pressing member according to Embodiment 1;

FIGS. 3D to 3F are a plan view, a side view, and a rear view, respectively, each showing a configuration of a fixing member according to Embodiment 1;

FIGS. 3G to 3I are a plan view, a side view, and a rear view, respectively, each showing a configuration of a second pressing member according to Embodiment 1;

FIGS. 4A and 4B are each a perspective view describing a process of attaching a screw onto a receiving member according to Embodiment 1;

FIG. 5A shows a state where the first pressing member is attached to the base member according to Embodiment 1;

FIGS. 5B and 5C each show a state where the second pressing member is attached to the fixing member according to Embodiment 1;

FIGS. 6A and 6B are perspective views showing a state where the fixture according to Embodiment 1 has been assembled, viewed from the front side and the rear side, respectively;

FIGS. 7A and 7B each show one example of a method for attaching the fixing member according to Embodiment 1;

FIG. 8A is a schematic diagram describing a process of funnel chest correction by use of bars according to Embodiment 1;

FIG. 8B shows a state where the fixture according to Embodiment 1 is attached to end portions of the bars;

FIG. 9 is an exploded perspective view of a fixture according to Embodiment 2;

FIGS. 10A to 10C are a plan view, a side view, and a rear view, respectively, each showing a configuration of a base member according to Embodiment 2;

FIGS. 10D to 10F are a plan view, a side view, and a rear view, respectively, each showing a configuration of a fixing member according to Embodiment 2;

FIGS. 11A and 11B are perspective views showing a state where the fixture according to Embodiment 2 has been assembled, viewed from the front side and the rear side, respectively;

FIG. 12 is an exploded perspective view of a fixture according to Embodiment 3;

FIGS. 13A to 13C are a plan view, a side view, and a rear view, respectively, each showing a configuration of a base member according to Embodiment 3;

FIGS. 13D to 13F are a plan view, a side view, and a rear view, respectively, each showing a configuration of a pressing member according to Embodiment 3;

FIGS. 14A and 14B are perspective views showing a state where the fixture according to Embodiment 3 has been assembled, viewed from the front side and the rear side, respectively;

FIG. 15 is an exploded perspective view of a fixture according to Embodiment 4;

FIG. 16A is a side view showing a state where members according to Embodiment 4 have been assembled;

FIG. 16B is a plan view of a base member according to Embodiment 4;

FIG. 16C is a rear view of a pressing member according to Embodiment 4;

FIG. 17 shows a state where the fixture according to Embodiment 4 is attached to end portions of bars;

FIG. 18 is an exploded perspective view of a fixture when another spacer is used, according to Embodiment 4;

FIG. 19 shows a state where the fixture is attached to the end portions of bars with use of another spacer, according to Embodiment 4; and

FIG. 20 shows a state where the fixture is attached to the end portions of bar without using a spacer, according to Embodiment 4.

It should be noted that these drawings are solely for description and do not limit the scope of the present invention by any degree.

### DESCRIPTION OF THE PREFERRED EMBODIMENTS

In the following, embodiments of the present invention will be described with reference to the drawings.

<Embodiment 1>

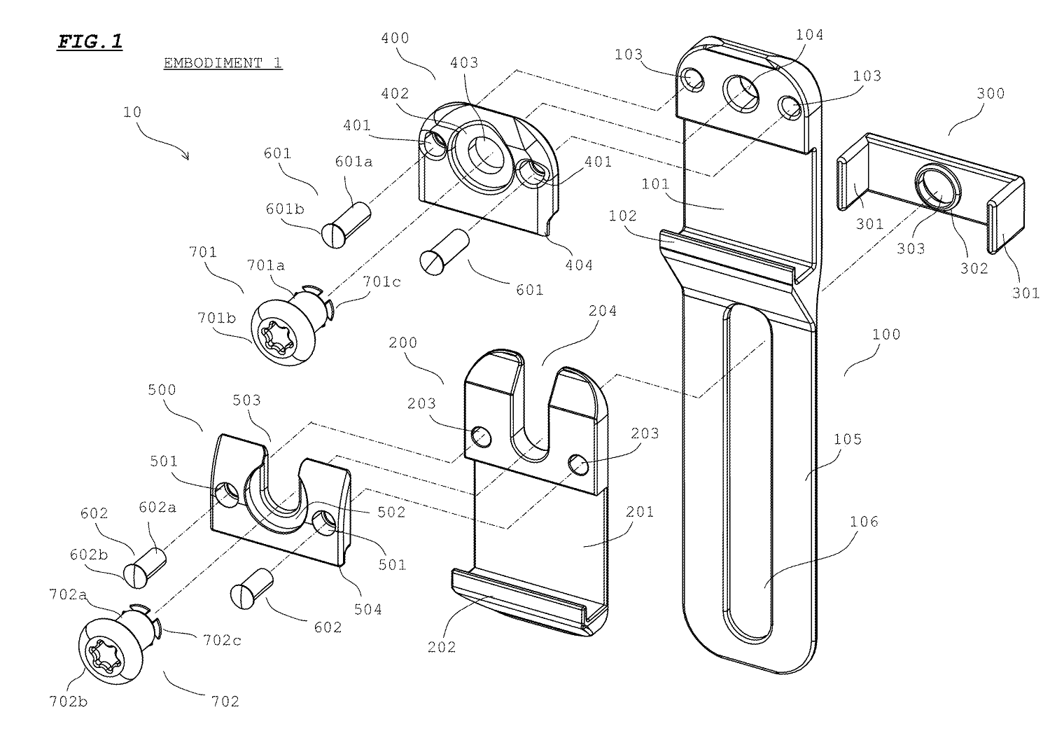

FIG. 1 is an exploded perspective view of a fixture 10 according to Embodiment 1.

The fixture 10 includes a base member 100, a fixing member 200, a receiving member 300, a first pressing member 400, a second pressing member 500, two pins 601, 602, and screws 701, 702. Each of these members is formed from a material, such as titanium, that has good affinity to human bodies and that can ensure rigidity.

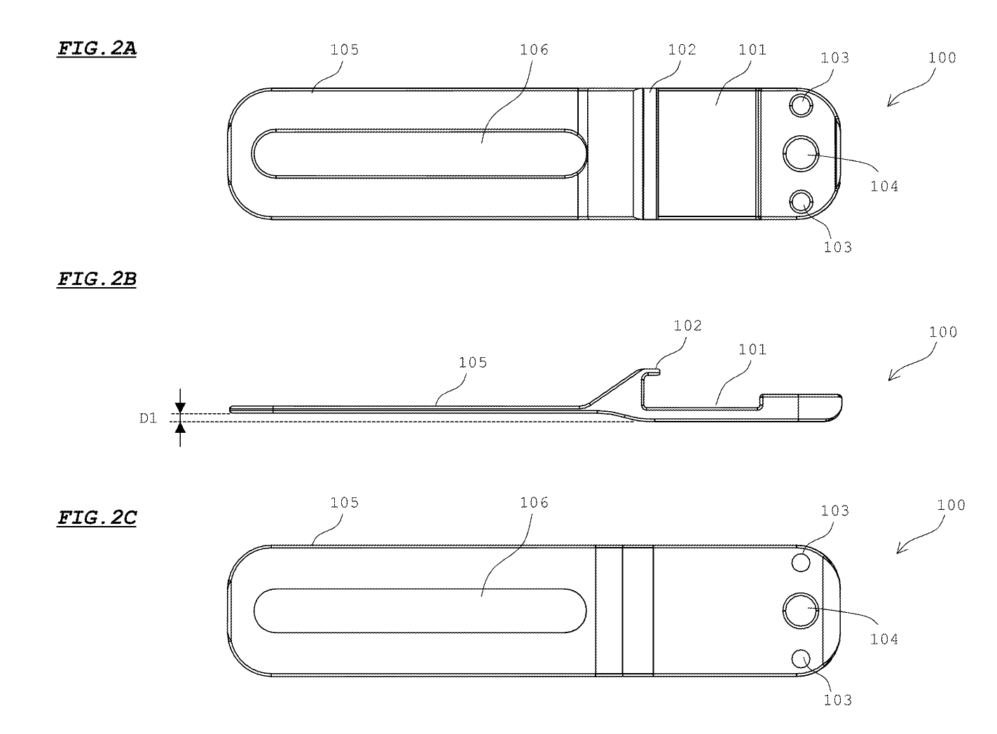

FIGS. 2A to 2C are a plan view, a side view, and a rear view, respectively, each showing a configuration of the base member 100.

With reference to FIG. 1 and FIGS. 2A to 2C, the base member 100 has, in a plan view, a contour obtained by rounding corners of a rectangle. The base member 100 has a shape that is symmetric with respect to the width direction, i.e., a direction orthogonal to the longitudinal direction. The base member 100 has a length that allows the base member 100 to extend across a plurality of ribs.

A groove portion 101 extending in a direction orthogonal to the longitudinal direction of the base member 100 is formed in the upper face of the base member 100. The groove portion 101 is for having a bar for funnel chest correction to be fitted therein. Therefore, the width of the groove portion 101 is slightly greater than the width of the bar. Two holes 103 into which the pins 601 are pressed are formed to a lateral side of the groove portion 101. A screw hole 104 into which the screw 701 is screwed is formed at an intermediate position between the two holes 103.

A flange portion 102 projecting inwardly toward the groove portion 101 from the upper face of the base member 100 is formed at the boundary, on the side opposite to the screw hole 104, of the groove portion 101. The distance between the bottom face of the groove portion 101 and the lower face of the flange portion 102 is substantially the same as the thickness of the bar fitted in the groove portion 101. In a state where the bar is fitted in the groove portion 101, the lower face of the flange portion 102 is at a height substantially the same as the upper face of the bar. The flange portion 102 is for preventing the bar from coming off, by lightly pressing the upper face of the bar fitted in the groove portion 101.

A support portion 105 extending in the longitudinal direction of the base member 100 is formed on the side opposite to the screw hole 104 with respect to the groove portion 101. The support portion 105 has a thin plate shape. Therefore, a user can deflect the support portion 105 into a direction perpendicular to the upper face of the base member 100 as appropriate, thereby causing the base member 100 to extend along the muscle layer. A long hole 106 extending in the longitudinal direction of the base member 100 is formed at the center in the width direction of the support portion 105.

The lower face of the support portion 105 is shifted upwardly by a height D1. The height D1 is the same as the thickness of the receiving member 300.

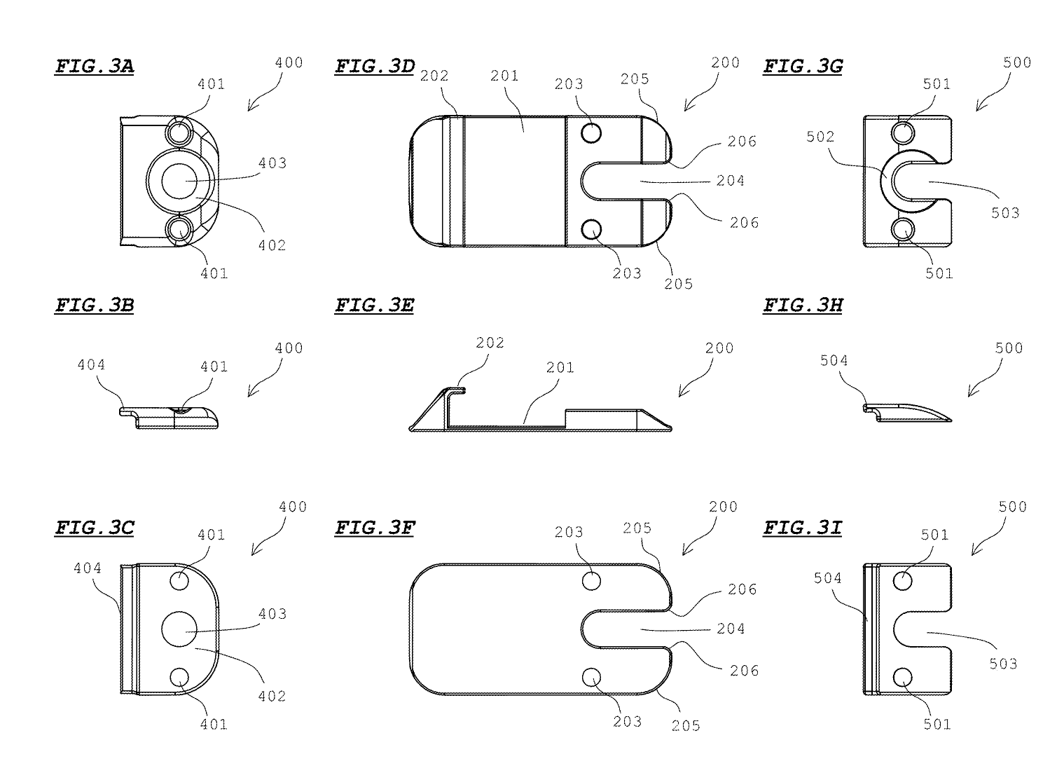

FIGS. 3D to 3F are a plan view, a side view, and a rear view, respectively, each showing a configuration of the fixing member 200.

With reference to FIG. 1 and FIGS. 3D to 3F, the fixing member 200 has, in a plan view, a contour obtained by recessing one side of a shape that is obtained by rounding corners of a rectangle. The fixing member 200 has a shape that is symmetric with respect to the width direction, i.e., a direction orthogonal to the longitudinal direction. The width of the fixing member 200 is substantially the same as the width of the support portion 105 of the base member 100.

A groove portion 201 extending in a direction orthogonal to the longitudinal direction of the fixing member 200 is formed at the upper face of the fixing member 200. The groove portion 201 is for having the bar for funnel chest correction to be fitted therein. Therefore, the width of the groove portion 201 is slightly greater than the width of the bar. Two holes 203 into which the pins 602 are pressed are formed to a lateral side of the groove portion 201. A cut groove 204 through which the screw 702 is passed is formed at an intermediate position between the two holes 203.

Two corner portions at the outer side of the end portion on the cut groove 204 side of the fixing member 200 are formed as smoothly-rounded inclined surfaces 205. Two corner portions at the inlet of the cut groove 204 are also formed as smoothly-rounded inclined surfaces 206.

A flange portion 202 projecting inwardly toward the groove portion 201 from the upper face of the fixing member 200 is formed at the boundary, on the side opposite to the cut groove 204, of the groove portion 201. The distance between the bottom face of the groove portion 201 and the lower face of the flange portion 202 is substantially the same as the thickness of the bar fitted in the groove portion 201. In a state where the bar is fitted in the groove portion 201, the lower face of the flange portion 202 is at a height substantially the same as the upper face of the bar. The flange portion 202 is for preventing the bar from coming off, by lightly pressing the upper face of the bar fitted in the groove portion 201.

With reference to FIG. 1, the receiving member 300 has a shape obtained by evenly bending both ends of a rectangular plate-like member. A pair of wall portions 301 opposed to each other are formed in the receiving member 300. The distance between the inner surfaces of the pair of wall portions 301 is slightly greater than the width of the support portion 105 of the base member 100 and the width of the fixing member 200. The pair of wall portions 301 are configured to be able to sandwich, in a direction orthogonal to the longitudinal direction of the base member 100, the fixing member 200 overlaid on the support portion 105, together with the support portion 105.

A ring-shaped protrusion 302 is formed at an intermediate position between the pair of wall portions 301. The inner surface of the protrusion 302 serves as a screw hole 303. The outer diameter of the protrusion 302 is substantially the same as the width of the long hole 106 of the base member 100. In addition, the inner diameter of the protrusion 302 is substantially the same as the width of the cut groove 204 of the fixing member 200. The screw 702 is screwed into the screw hole 303. The receiving member 300 has a shape symmetric with respect to the axis penetrating the center of the screw hole 303.

FIGS. 3A to 3C are a plan view, a side view, and a rear view, respectively, each showing a configuration of the first pressing member 400.

With reference to FIG. 1 and FIGS. 3A to 3C, in a plan view, the lower face of the first pressing member 400 has a contour that substantially overlaps an upper face region, of the base member 100, where the holes 103 and the screw hole 104 are formed. The first pressing member 400 has a shape symmetric with respect to the width direction.

Two holes 401 into which the pins 601 are inserted are formed in the first pressing member 400. A circular recessed portion 402 is formed at an intermediate position between the two holes 401, and further, a hole 403 through which the screw 701 is passed is formed at the center of the recessed portion 402. A flange portion 404 projecting from the upper face of the first pressing member 400 is formed at the boundary on the side opposite to the hole 403.

FIGS. 3G to 3I are a plan view, a side view, and a rear view, respectively, each showing a configuration of a second pressing member 500.

With reference to FIG. 1 and FIGS. 3G to 3I, in a plan view, the lower face of the second pressing member 500 has a contour that substantially overlaps an upper face region, of the fixing member 200, where the holes 203 and the cut groove 204 are formed. The second pressing member 500 has a shape symmetric with respect to the width direction.

Two holes 501 into which the pins 602 are inserted are formed in the second pressing member 500. A circular recessed portion 502 is formed at an intermediate position between the two holes 501, and further, a cut groove 503 through which the screw 702 is passed is formed at the center in the width direction of the recessed portion 502. A flange portion 504 projecting from the upper face of the second pressing member 500 is formed at the boundary on the side opposite to the cut groove 503.

Each pin 601 includes a shank 601a having a cylindrical shape, and a head 601b having a greater diameter than the shank 601a. The diameter of the shank 601a is slightly greater than the diameter of the hole 103 of the base member 100. Each hole 401 of the first pressing member 400 includes a large-diameter portion on the upper face side, and a small-diameter portion on the lower face side. The diameter of the large-diameter portion is slightly greater than the diameter of the small-diameter portion. The diameter of the shank 601a of the pin 601 is slightly smaller than the diameter of the small-diameter portion of the hole 401. In addition, the diameter of the head 601b of the pin 601 is smaller than the diameter of the large-diameter portion of the hole 401, and is greater than the diameter of the small-diameter portion thereof. Therefore, when the pin 601 is inserted into the hole 401, the shank 601a passes through the hole 401 but the head 601b is caught by the small-diameter portion of the hole 401.

Similarly, each pin 602 includes a shank 602a having a cylindrical shape, and a head 602b having a greater diameter than the shank 602a. The diameter of the shank 602a is slightly greater than the diameter of the hole 203 of the fixing member 200. Each hole 501 of the second pressing member 500 includes a large-diameter portion on the upper face side, and a small-diameter portion on the lower face side. The diameter of the shank 602a of the pin 602 is slightly smaller than the diameter of the small-diameter portion of the hole 501. In addition, the diameter of the head 602b of the pin 602 is smaller than the diameter of the large-diameter portion of the hole 501, and is greater than the diameter of the small-diameter portion thereof. Therefore, when the pin 602 is inserted into the hole 501, the shank 602a passes through the hole 501 but the head 602b is caught by the small-diameter portion of the hole 501.

The screw 701 includes a screw portion 701a having a thread groove formed therein, and an umbrella-shaped head 701b. Further, at an end of the screw portion 701a, the screw 701 includes a stopper 701c composed of four pieces that can be elastically deformed into directions toward the center axis of the screw 701. Similarly, the screw 702 also includes a screw portion 702a, a head 702b, and a stopper 702c. When the screws 701, 702 are inserted into the screw holes 104, 303, the stoppers 701c, 702c are locked to the rear face of the base member 100 and the rear face of the receiving member 300, thereby preventing the screws 701, 702 from coming off.

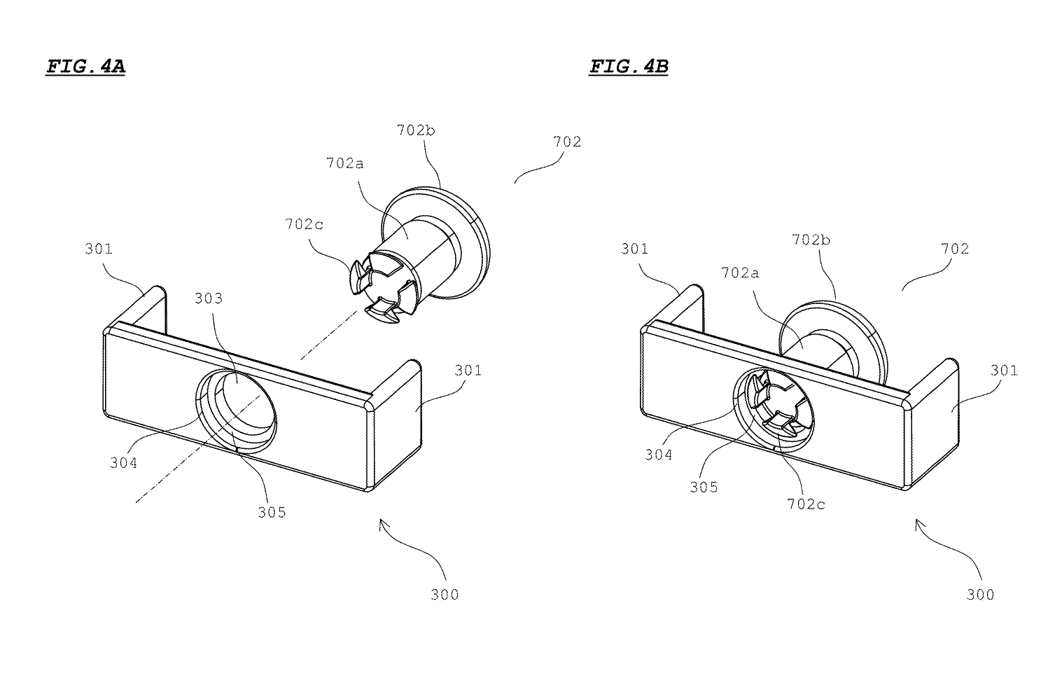

FIGS. 4A and 4B are each a perspective view describing a process of attaching the screw 702 to the receiving member 300.

As shown in FIG. 4A, at the rear side of the screw hole 303 of the receiving member 300, a hole 304 having a greater diameter than the screw hole 303 is formed. Accordingly, a step portion 305 is formed at the boundary between the hole 304 and the screw hole 303. When the screw portion 702a of the screw 702 is inserted into the screw hole 303, the stopper 702c is locked to the step portion 305.

That is, from the state shown in FIG. 4A, when the inclined surfaces of the four pieces of the stopper 702c are placed against the inlet of the screw hole 303 and the screw 702 is pushed thereinto, the four pieces elastically deform into directions toward the center axis of the screw 701, to be inside the inner surface of the screw hole 303. When the screw 702 is further pushed in, the four pieces pass the inner surface of the screw hole 303 and reach the hole 304. Associated with this, the four pieces of the stopper 702c elastically return to their original positions, and as shown in FIG. 4B, the four pieces are engaged with the step portion 305. As a result, the screw 702 is prevented from coming off from the screw hole 303.

In a state where the screw 702 is prevented from coming off as shown in FIG. 4B, the screw portion 702a is not meshed with the thread groove of the screw hole 303. When the screw 702 is rotated while being slightly pressed from the state shown in FIG. 4B, the screw portion 702a is meshed with the thread groove of the screw hole 303, whereby the screw 702 is screwed onto the receiving member 300.

Through a similar process to this, the screw 701 shown in FIG. 1 is also prevented from coming off from the screw hole 104 of the base member 100. It should be noted that the rear face at the position of the screw hole 104 of the base member 100 is not provided with structures corresponding to the hole 304 and the step portion 305 shown in FIG. 4A. Therefore, the stopper 701c of the screw 701 is locked to the rear face of the base member 100 around the screw hole 104. The rear face at the position of the screw hole 104 of the base member 100 may be provided with structures corresponding to the hole 304 and the step portion 305 shown in FIG. 4A.

When the members shown in FIG. 1 are to be assembled, first, the first pressing member 400 is overlaid on the region, of the upper face of the base member 100, where the holes 103 are formed. In this state, the shanks 601a of the two pins 601 are passed through the holes 401 of the first pressing member 400, and pressed into the holes 103 of the base member 100. Accordingly, the first pressing member 400 is supported by the base member 100.

Next, the second pressing member 500 is overlaid on the region, of the upper face of the fixing member 200, where the holes 203 are formed. In this state, the shanks 602a of the two pins 602 are passed through the holes 501 of the second pressing member 500, and pressed into the holes 203 of the fixing member 200. Accordingly, the second pressing member 500 is supported by the fixing member 200.

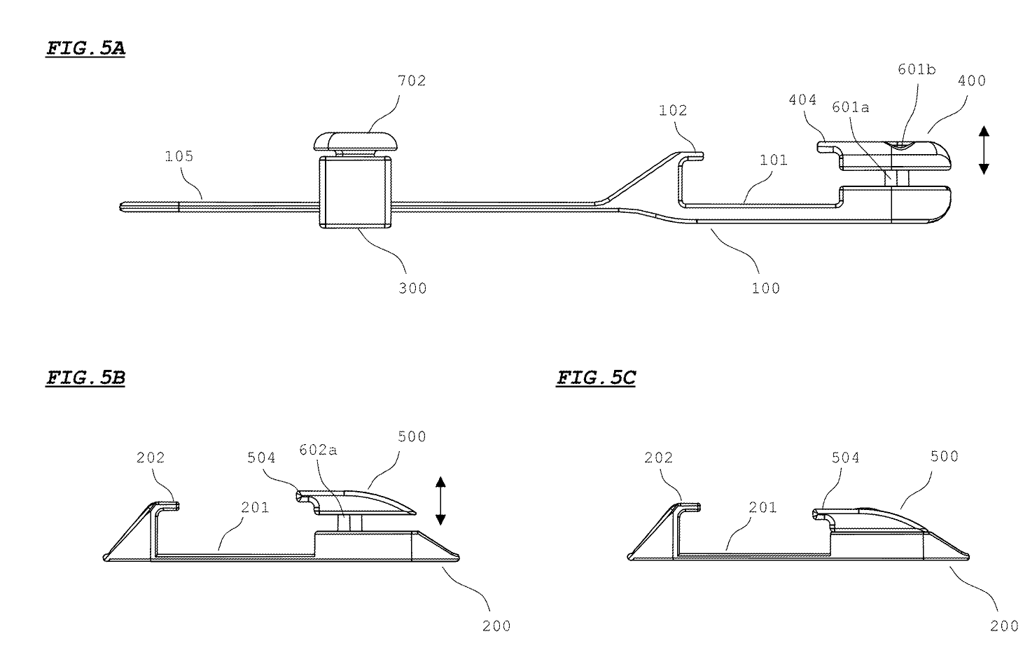

FIG. 5A shows a state where the first pressing member 400 is attached to the base member 100.

The length of the shank 601a of the pin 601 is greater than the thickness of the first pressing member 400. Therefore, as shown in FIG. 5A, the first pressing member 400 is movable upwardly in a range allowed by the length of the shank 601a, i.e., in a range up to where the movement of the first pressing member 400 is restricted by the head 601b of the pin 601. If the first pressing member 400 moves upwardly in this manner, the distance between the flange portion 404 of the first pressing member 400 and the bottom face of the groove portion 101 of the base member 100 is increased. Accordingly, the bar can be easily fitted into the groove portion 101.

FIGS. 5B and 5C each show a state where the second pressing member 500 is attached to the fixing member 200.

Similar to the first pressing member 400, the second pressing member 500 is also movable upwardly in a range allowed by the length of the shank 602a, i.e., in a range up to where the movement of the second pressing member 500 is restricted by the head 602b of pin 602. If the second pressing member 500 moves upwardly, the distance between the flange portion 504 of the second pressing member 500 and the bottom face of the groove portion 201 of the fixing member 200 is increased. Accordingly, the bar can be easily fitted into the groove portion 201.

With reference back to FIG. 1, next, the screw 701 is passed through the hole 403 of the first pressing member 400 and the screw hole 104 of the base member 100, and the screw 701 is prevented from coming off from the screw hole 104 as described above. As a result, the screw 701 is attached to the base member 100.

Further, the receiving member 300 is fitted to the support portion 105 from the rear side of the base member 100 such that the pair of wall portions 301 sandwich the support portion 105. At this time, the protrusion 302 of the receiving member 300 is engaged with the long hole 106 of the support portion 105. Since the height of the protrusion 302 is the same as the thickness of the support portion 105, the upper face of the protrusion 302 is positioned at the same plane as the upper face of the support portion 105.

Next, the screw 702 is passed through the screw hole 303 of the receiving member 300, and the screw 702 is prevented from coming off from the screw hole 303 as described above. Accordingly, the screw 702 is attached to the receiving member 300. Further, while the fixing member 200 is moved along the upper face of the support portion 105, the dead end of the cut groove 204 of the fixing member 200 and the dead end of the cut groove 503 of the second pressing member 500 are caused to be engaged with the screw portion 702a of the screw 702. In this state, the screw 702 is fastened. Accordingly, assembly of the fixture 10 is completed.

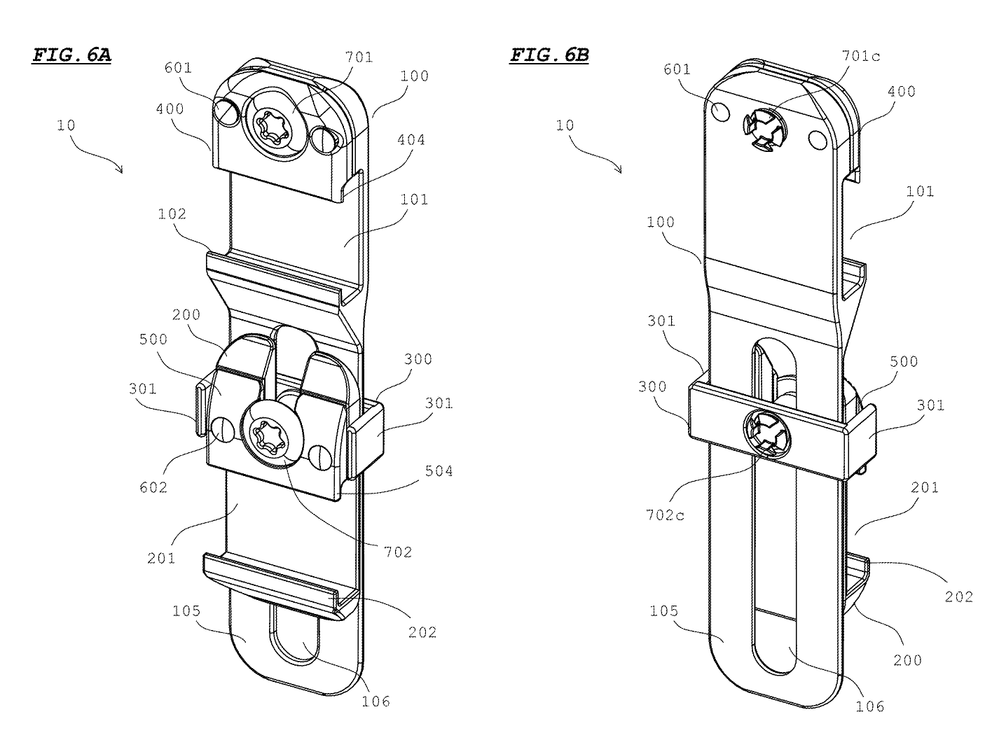

FIGS. 6A and 6B are perspective views showing a state where the fixture 10 has been assembled, viewed from the front side and the rear side, respectively. Here, a state where the screws 701, 702 have been completely fastened is shown.

In the case of the fixture 10 of Embodiment 1, bars can be fixed the groove portion 101 and the groove portion 201, respectively. For surgery, the screw 701 is loosened to produce a state where a bar can be attached to the groove portion 101. Further, the screw 702 is loosened to remove, from the base member 100, the fixing member 200 having the second pressing member 500 attached thereto.

Then, the base member 100 is located on the muscle layer near the end portion of the bar, and the end portion of the bar is fitted into the groove portion 101. In this case, the end portion of the bar is inserted, for example, from a lateral side of the groove portion 101 into the gap between the bottom face of the groove portion 101 and the flange portions 102, 404, thereby being fitted into the groove portion 101. Then, the base member 100 is shifted in a direction orthogonal to the longitudinal direction, so as to be located at a position on the muscle layer where the base member 100 is most stabilized. In this state, the screw 701 is fastened so that the upper face of the end portion of the bar is pressed by the flange portion 404. Accordingly, the end portion of the bar is pressed against the bottom face of the groove portion 101, and the end portion of the bar is fixed to the base member 100.

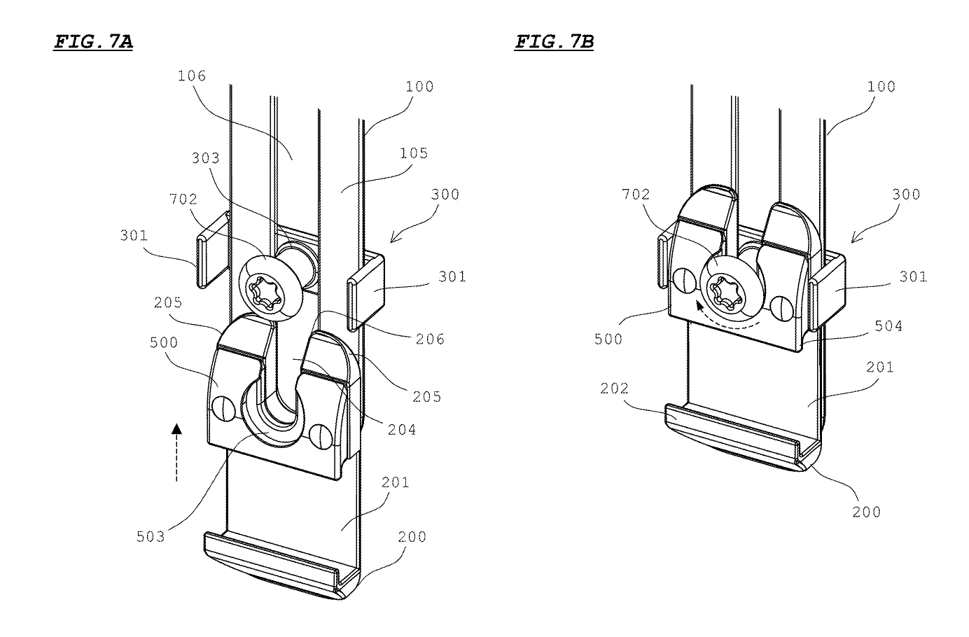

Next, an end portion of another bar is fitted into the groove portion 201 of the fixing member 200. At this time, the end portion of the bar is prevented from coming off from the groove portion 201, by the flange portion 202 and the flange portion 504. Then, as shown in FIGS. 7A and 7B, while the fixing member 200 is moved along the upper face of the support portion 105, the dead end of the cut groove 204 of the fixing member 200 and the dead end of the cut groove 503 of the second pressing member 500 are caused to be engaged with the screw portion 702a of the screw 702. At this time, the fixing member 200 is sandwiched by the pair of wall portions 301 of the receiving member 300. Then, the fixing member 200 is moved in the longitudinal direction together with the receiving member 300, so that the fixing member 200 and the receiving member 300 are located at a position where the bar can be stably secured. In this state, the screw 702 is fastened and the end portion of the other bar is fixed to the fixing member 200. At this time, the fixing member 200 is simultaneously fixed to the base member 100.

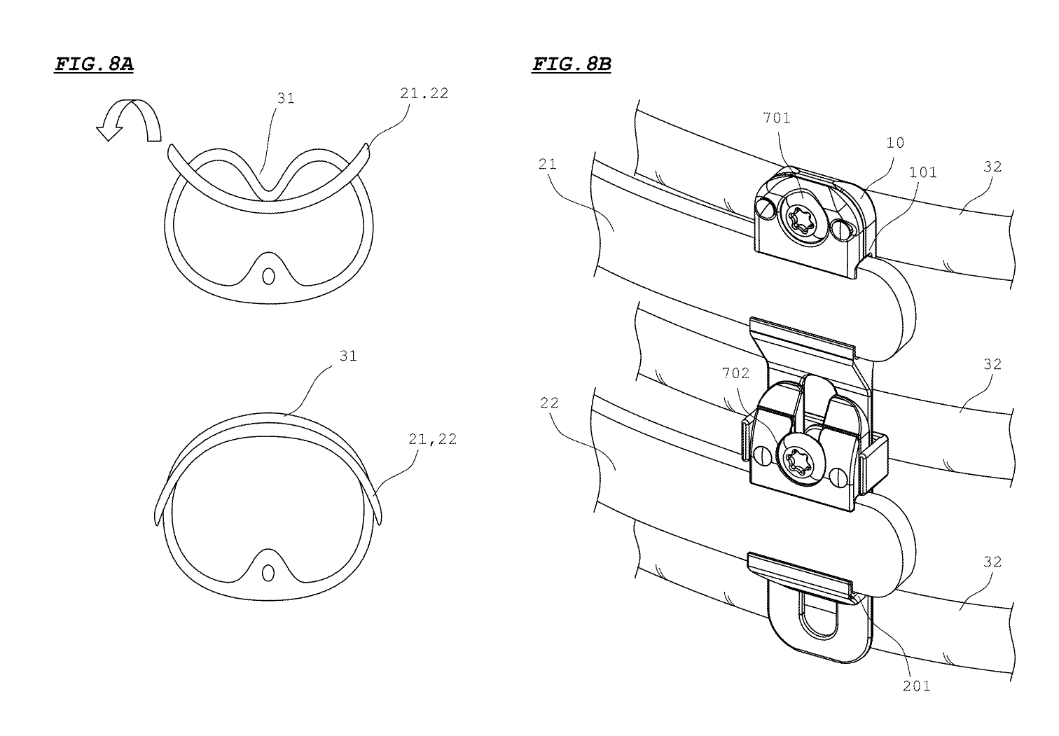

FIG. 8A is a schematic diagram describing a process of funnel chest correction by use of bars 21, 22.

In funnel chest correction surgery, the bars 21, 22 made of stainless and having an arch shape are inserted into the body, and then, the sternum 31 is pushed up by the bars 21, 22 from inside the body, whereby funnel chest correction is performed.

Specifically, incisions are made at both sides of a patient in order to pass the arch-shaped bars 21, 22, and each bar 21, 22 is inserted into the thoracic cavity under the muscle layer from a portion between ribs where the intercostal muscle is detached (see the upper part of FIG. 8A). Accordingly, a center portion of the bar 21, 22 is located inside the sternum 31 of the patient. At this time, both ends of the bar 21, 22 are in a state of protruding from both sides of the patient. In this state, the bar 21, 22 is turned by 180 degrees, whereby the sternum 31 of the patient is pushed up by the bar 21, 22 (see the lower part of FIG. 8A). Then, as described above, the fixture 10 is attached to both ends of the bar 21, 22, whereby both ends of the bar 21, 22 are fixed.

FIG. 8B shows a state where the fixture 10 is attached to end portions of the bars 21, 22.

As shown in FIG. 8B, the fixture 10 is set above a plurality of ribs 32 so as to extend across the ribs 32. For convenience, FIG. 8B shows the fixture 10 as being placed on the ribs 32. However, in actuality, the fixture 10 is placed on the muscle layer between the ribs 32 and the skin. The bars 21, 22 are respectively fitted in the groove portions 101, 201 of the fixture 10. The bars 21, 22 are fixed to the fixture 10 as a result of the screws 701, 702 being fastened.

<Effects of Embodiment 1>

According to Embodiment 1, the following effects can be exhibited.

End portions of the bars 21, 22 can be fixed to the fixture 10 through simple operations such as fitting the end portions of the bars 21, 22 into the groove portions 101, 201, setting the base member 100 to the outside of a plurality of ribs 32 so as to extend across the ribs 32, and fastening the screws 701, 702. In addition, the end portions of the bars 21, 22 are slidable with respect to the groove portions 101, 201. Therefore, in a state where the end portions of the bars 21, 22 are fitted in the groove portions 101, 201, the fixture 10 can be located at a position on the muscle layer where the fixture 10 is most stabilized. If the screws 701, 702 are fasted in this state, the end portions of the bars 21, 22 can be fixed to the fixture 10 at this position. Therefore, according to Embodiment 1, the bars 21, 22 can be stably secured to the chest of a patient through simple operations.

The fixture 10 of Embodiment 1 is configured to use the first pressing member 400 and the second pressing member 500 as a pressing part that presses the bars 21, 22. Therefore, a greater area of the upper faces of the bars 21, 22 can be pressed by the first pressing member 400 and the second pressing member 500, and the end portions of the bars 21, 22 can be more stably fixed.

In addition, the upper faces of the bars 21, 22 are pressed by the first pressing member 400 and the second pressing member 500 from one side of the groove portions 101, 201, and at the other side of the groove portions 101, 201, the upper faces of the bars 21, 22 are pressed by the flange portions 102, 202. Therefore, compared with a configuration in which both sides of the groove portions 101, 201 are respectively pressed by pressing members, a simplified configuration can be realized, and the man-hour for fastening screws can be reduced. Accordingly, the operation of fixing the bars 21, 22 can be more simplified.

In addition, the first pressing member 400 and the second pressing member 500 are configured to be supported by the base member 100 and the fixing member 200 by means of the pins 601, 602, such that the first pressing member 400 and the second pressing member 500 are prevented from coming off from the base member 100 and the fixing member 200 and are allowed to be close to and separated from the groove portions 101, 201. Therefore, it is not necessary to separately bring and overlay the first pressing member 400 and the second pressing member 500 to the base member 100 and the fixing member 200, respectively. Accordingly, the operation of fixing the bars 21, 22 can be more simplified.

Further, as shown in FIG. 8B, the fixture 10 of Embodiment 1 can fix the end portions of the two bars 21, 22, simultaneously. In addition, in a state where the fixture 10, long in the longitudinal direction, bridges the end portions of the two bars 21, 22, the fixture 10 is set above the ribs. This can further assuredly prevent rotation of the two bars 21, 22 from the state shown in the lower part of FIG. 8A to the state shown in the upper part of FIG. 8A. Thus, the two bars 21, 22 can be secured to the chest of the patient in a very stable manner.

Further, since the position of the fixing member 200 can be changed along the long hole 106, the position of the fixing member 200 can be adjusted in accordance with the interval between the two bars 21, 22 set in the patient. Therefore, the two bars 21 22 can be secured, with the groove portion 101 of the base member 100 and the groove portion 201 of the fixing member 200 located at the optimum positions for the state of the ribs 32 of the patient. In addition, when the screw 702 is fastened, the bar 22 is fixed to the fixing member 200, and simultaneously, the fixing member 200 is fixed to the base member 100. Therefore, the operation of fixing the bar 22 can be performed very easily.

Further, the receiving member 300 is configured to sandwich, by the pair of wall portions 301, the fixing member 200 overlaid on the support portion 105 of the base member 100, together with the support portion 105. Therefore, using the receiving member 300 as a guide, the fixing member 200 can be easily moved along the support portion 105 in the longitudinal direction of the base member 100.

Further, the fixing member 200 includes the cut groove 204 that allows the screw 702 to pass therethrough. Thus, as shown in FIGS. 7A, 7B, if the cut groove 204 of the fixing member 200 is fitted to the screw 702 from a lateral side in a state where the screw 702 is attached in a temporary manner to the receiving member 300, the fixing member 200 can be located between the screw 702 and the receiving member 300. Accordingly, the operation of attaching the fixing member 200 to the base member 100 can be easily performed.

Here, the inclined surfaces 205, 206 are formed in the end portion on the cut groove 204 side of the fixing member 200. Therefore, if the fixing member 200 is caused to slide as shown in FIG. 7A, the fixing member 200 smoothly enters between the pair of wall portions 301 of the receiving member 300 by being guided by the inclined surfaces 206, and the cut groove 204 is smoothly engaged with the screw portion 702a of the screw 702 by being guided by the inclined surfaces 206. Therefore, the fixing member 200 can be easily located to the position shown in FIG. 7B from the position shown in FIG. 7A.

It should be noted that the fixture 10 of Embodiment 1 is used in combination with the bars 21, 22 for funnel chest correction, to form a funnel chest correction device. The number of bars included in the funnel chest correction device is not limited to two, and may be one, three, or greater. The thicknesses of the bars that can be fitted in the groove portions 101, 201 may not necessarily be the same with each other. The shapes and dimensions of the members of the fixture 10 may be adjusted such that bars having different thicknesses can be fitted in the groove portions 101, 201.

The method for attaching the fixing member 200 to the base member 100 at the time of surgery is not necessarily limited to the method described above, and various other methods can be employed. For example, the screw 702 need not be previously fixed in a temporary manner to the receiving member 300, and may be apart from the receiving member 300. In this case, for example, in a state where the end portion of the bar 22 is fitted in the groove portion 201, the fixing member 200 and the receiving member 300 are caused to slide along the support portion 105 to locate the fixing member 200 to a desired position, and then, the screw 702 is pressed into the screw hole 303 of the receiving member 300 from above via the cut groove 204, whereby the screw 702 may be fastened.

However, in this case, the receiving member 300 is not attached in a temporary manner to the base member 100, either. Therefore, during surgery, an operation of fitting the receiving member 300 to the support portion 105 of the base member 100 from the rear side, and an operation of pressing the screw 702 from the cut groove 204 into the screw hole 303 are required. In contrast, as described above, if the screw 702 is previously attached in a temporary manner to the screw hole 303 of the receiving member 300 in a state where the receiving member 300 is fitted to the support portion 105, these operations are not required during surgery. Thus, the surgery can be performed smoothly.

<Embodiment 2>

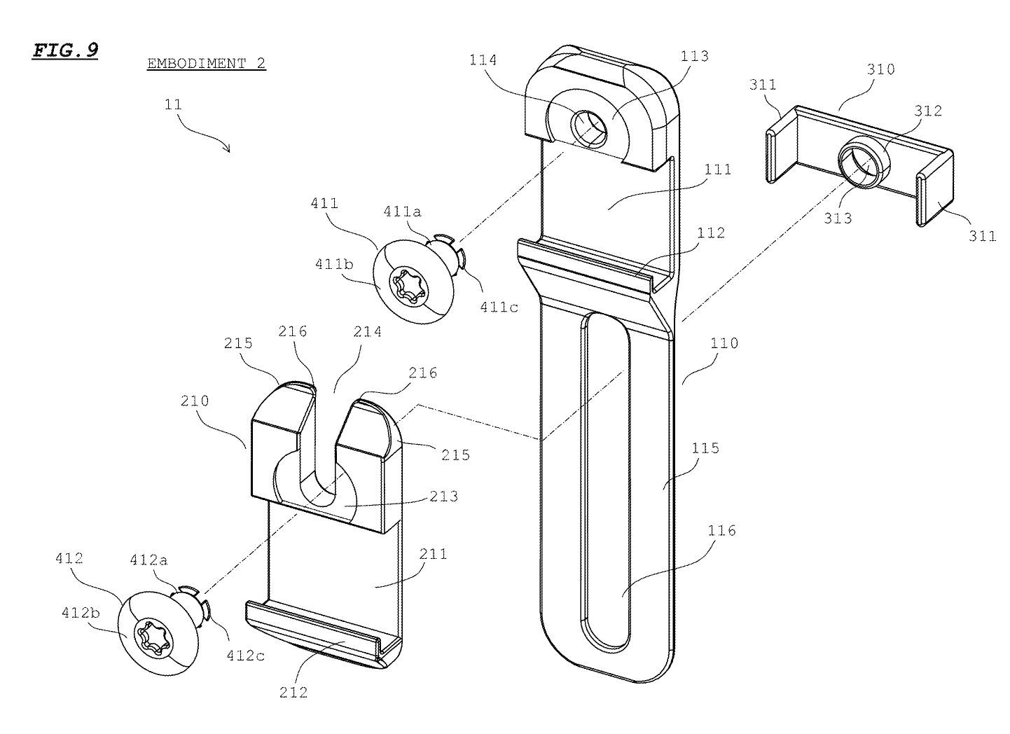

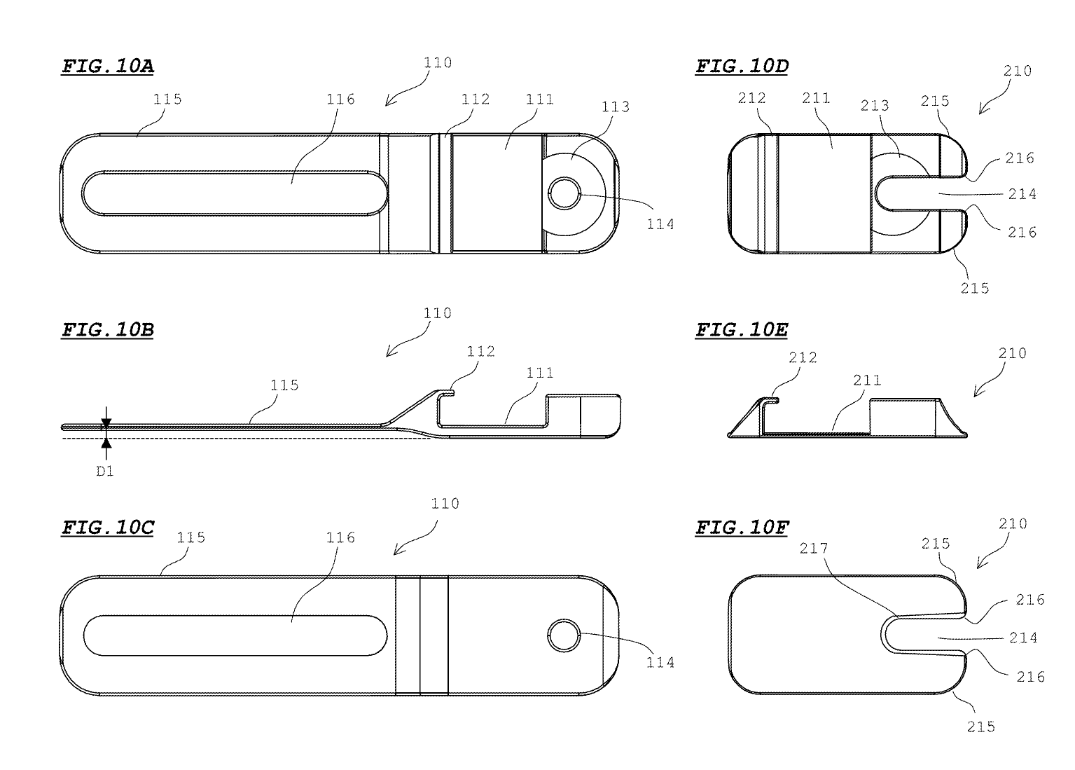

FIG. 9 is an exploded perspective view of a fixture 11 according to Embodiment 2. FIGS. 10A to 10C are a plan view, a side view, and a rear view, respectively, each showing a configuration of a base member 110 according to Embodiment 2. FIGS. 10D to 10F are a plan view, a side view, and a rear view, respectively, each showing a configuration of a fixing member 210 according to Embodiment 2.

In Embodiment 2, the first pressing member 400, the second pressing member 500, and the pins 601, 602 in Embodiment 1 are omitted. The bars 21, 22 are pressed by heads 411b, 412b of screws 411, 412, respectively.

As shown in FIG. 9, the fixture 11 includes the base member 110, the fixing member 210, a receiving member 310, and the screws 411, 412.

With reference to FIG. 9 and FIGS. 10A to 10C, in the base member 110, the thickness of the part where a screw hole 114 is formed is greater than that of the corresponding part of the base member 100 of Embodiment 1. In addition, around the screw hole 114, a recessed portion 113 having a diameter slightly smaller than that of the head 411b of the screw 411 is formed. The groove portion 111 side of the recessed portion 113 is continuous with the groove portion 111. The depth of the recessed portion 113 is slightly smaller than the thickness of the head 411b of the screw 411. The screw hole 114 is provided at a position close to the groove portion 111. The configurations of the groove portion 111, a flange portion 112, a support portion 115, and a long hole 116 are the same as those of the groove portion 101, the flange portion 102, the support portion 105, and the long hole 106 of Embodiment 1, respectively.

With reference to FIG. 9 and FIGS. 10D to 10F, in the fixing member 210, the thickness of the part where a cut groove 214 is formed is greater than that of the corresponding part of the fixing member 200 of Embodiment 1. In addition, around the dead end of the cut groove 214, a recessed portion 213 having a diameter slightly smaller than that of the head 412b of the screw 412 is formed. The groove portion 211 side of the recessed portion 213 is continuous with the groove portion 211. The cut groove 214 extends to a position close to the groove portion 211.

On the rear face of the fixing member 210 and around the cut groove 214, a guide groove 217 that is recessed with respect to the other part of the rear face is formed. The guide groove 217 is to be engaged with the top portion of a protrusion 312 of the receiving member 310 to guide the protrusion 312. The width of the guide groove 217 is gradually increased toward the inlet of the cut groove 214 so that the top portion of the protrusion 312 can be smoothly guided to the dead end of the cut groove 214.

The configurations of the groove portion 211, a flange portion 212, and inclined surfaces 215, 216 are the same as those of the groove portion 201, the flange portion 202, and the inclined surfaces 205, 206 of Embodiment 1, respectively.

With reference to FIG. 9, in the receiving member 310, the height of the protrusion 312 is greater than that of the protrusion 302 of Embodiment 1. Therefore, when the receiving member 310 is fitted to the support portion 115 of the base member 110, the top portion of the protrusion 312 protrudes from the upper face of the support portion 115. The protruding top portion of the protrusion 312 is engaged with the guide groove 217 on the rear face of the fixing member 210 overlaid on the upper face of the support portion 115. The configurations of wall portions 311, a screw hole 313, and the rear side of the receiving member 310 are the same as those of the receiving member 300 of Embodiment 1.

In the screws 411, 412, the diameters of the heads 411b, 412b are greater than the diameters of the heads 701b, 702b of the screws 701, 702 of Embodiment 1. The configurations of screw portions 411a, 412a and stoppers 411c, 412c are the same as those of the screw portions 701a, 702a and the stoppers 701c, 702c of the screws 701, 702 of Embodiment 1.

When the fixing member 210 is to be attached to the base member 110, first, the receiving member 310 is fitted to the support portion 115 from the rear side of the support portion 115. At this time, the protrusion 312 of the receiving member 310 passes through the long hole 116 and protrudes from the upper face of the support portion 115. In this state, as in the case of Embodiment 1, the screw 412 is pressed into the screw hole 313, whereby the screw 412 is attached in a temporary manner to the screw hole 313. Further, while the fixing member 210 is moved along the upper face of the support portion 115, the dead end of the cut groove 214 of the fixing member 210 is caused to be engaged with the screw portion 412a of the screw 412. In this state, the screw 412 is fastened. Further, the screw 411 is pressed into the screw hole 114, whereby the screw 411 is attached in a temporary manner to the screw hole 114. Accordingly, assembly of the fixture 11 is completed.

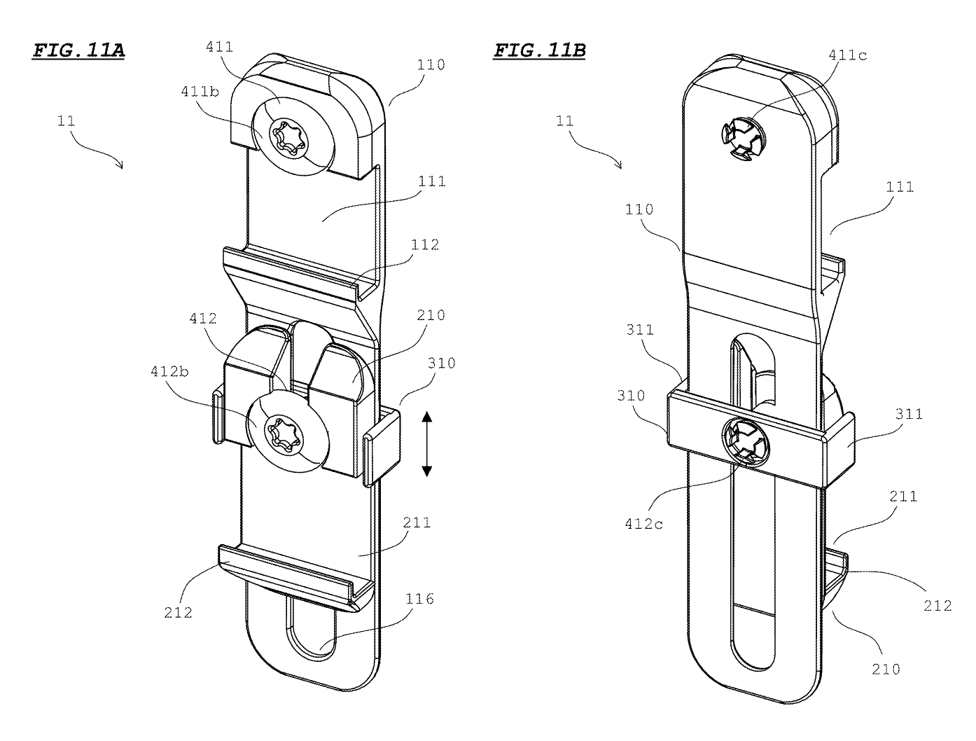

FIGS. 11A, 11B are perspective views showing a state where the fixture 11 has been assembled, viewed from the front side and the rear side, respectively. Here, a state where the screws 411, 412 have been fastened is shown.

As shown in FIGS. 11A, 11B, the heads 411b, 412b of the screws 411, 412 extend into the groove portions 111, 211. In Embodiment 2, the bars 21, 22 fitted in the groove portions 111, 211 are pressed by the parts of the heads 411b, 412b extending into the groove portions 111, 211. That is, in a state where the screws 411, 412 are loosened (temporarily attached state), the bars 21, 22 are fitted into the groove portions 111, 211, and then, the screws 411, 412 are fastened. Accordingly, the upper faces of the bars 21, 21 are pressed by the lower faces of the heads 411b, 412b, whereby the bars 21, 22 are pressed against the bottom faces of the groove portions 111, 211. As a result, the end portions of the bars 21, 22 are fixed to the fixture 11. The method for setting the fixture 11 during surgery is the same as that according to Embodiment 1 described above.

<Effects of Embodiment 2>

According to Embodiment 2, effects similar to those of Embodiment 1 can be exhibited.

In Embodiment 2, the bars 21, 22 are pressed by the heads 411b, 412b of the screws 411, 412. Therefore, unlike Embodiment 1, it is not necessary to separately provide members for pressing the upper faces of the bars 21, 22 (the first pressing member 400, the second pressing member 500). Therefore, the configuration of the fixture 11 can be simplified.

However, in Embodiment 2, the areas of the upper faces of the bars 21, 22 pressed by the heads 411b, 412b of the screws 411, 412 are smaller than those in the case of Embodiment 1. Therefore, in order to stabilize the fixation of the bars 21, 22 by pressing greater areas, the upper faces of the bars 21, 22 are preferably pressed by the first pressing member 400 and the second pressing member 500 as in Embodiment 1.

In Embodiment 2, the recessed portions 113, 213 into which the heads 411b, 412b of the screws 411, 412 are fitted and which are continuous with the groove portions 111, 211 are formed around the screw hole 114 and the cut groove 214. Therefore, the heads 411b, 412b of the screws 411, 412 can be prevented from significantly protruding from the upper faces of the base member 110 and the fixing member 210 when the screws 411, 412 are fastened. In addition, after the screws 411, 412 are fastened and fixed, the heads 411b, 412b of the screws 411, 412 are less likely to rub the affected area. Further, the heads 411b, 412b of the screws 411, 412 can be caused to partially extend above the groove portions 111, 211 from the recessed portions 113, 213, and the bars 21, 22 can be appropriately pressed by the heads 411b, 412b of the screws 411, 412.

The screw hole 114 is provided only on one side in the longitudinal direction of the base member 110 with respect to the groove portion 111. The flange portion 112 projecting inwardly toward the groove portion 111 from the boundary on the other side in the longitudinal direction of the groove portion 111 is provided to the base member 110. While a part on the other side of the end portion of the bar 21 is prevented from coming off by the flange portion 112, the upper face on the one side of the end portion of the bar 21 is pressed by the head 411b of the screw 411, whereby the end portion is fixed to the groove portion 111. Accordingly, the number of screws with which fastening operation is performed can be reduced, and thus, the fixing operation can be significantly simplified.

The depths of the recessed portions 113, 213 may be set to depths that allow the heads 411b, 412b to be completely buried in the recessed portions 113, 213 in a state where the screws 411, 412 are fastened. The height of the protrusion 312 of the receiving member 310 may be set to be the same as that of the protrusion 302 of Embodiment 1 so that the top portion of the protrusion 312 does not protrude from the upper face of the support portion 115 in a state where the receiving member 310 is fitted to the support portion 115.

<Embodiment 3>

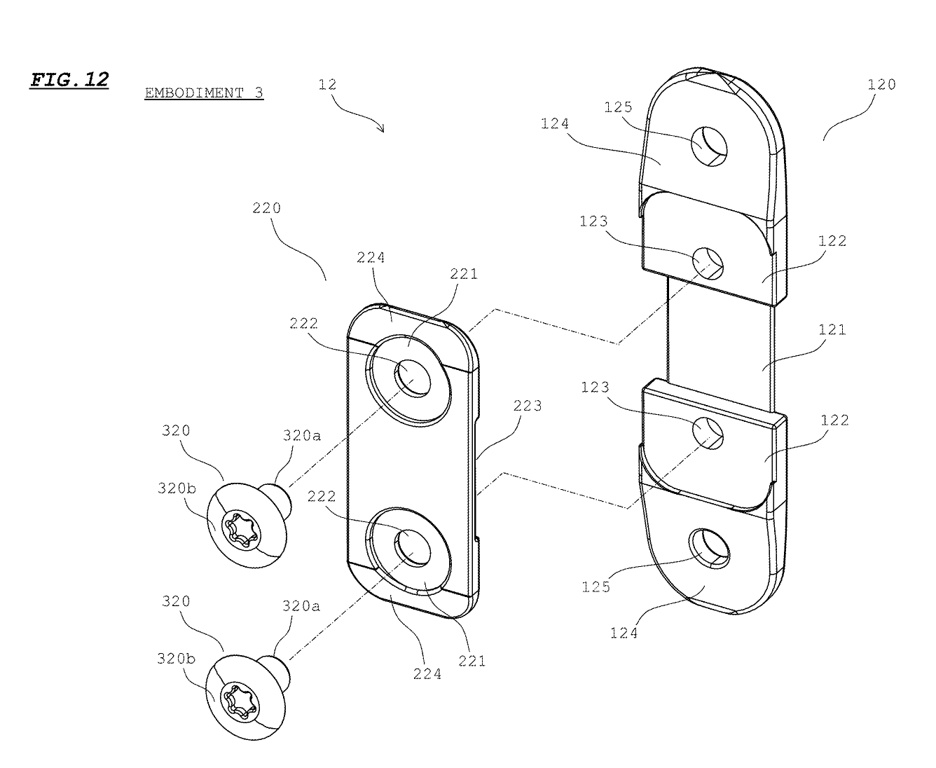

FIG. 12 is an exploded perspective view of a fixture 12 according to Embodiment 3.

The fixture 12 includes a base member 120, a pressing member 220, and screws 320. Similar to Embodiments 1, 2 above, each member is formed from a material, such as titanium, that has good affinity to human bodies and that can ensure rigidity.

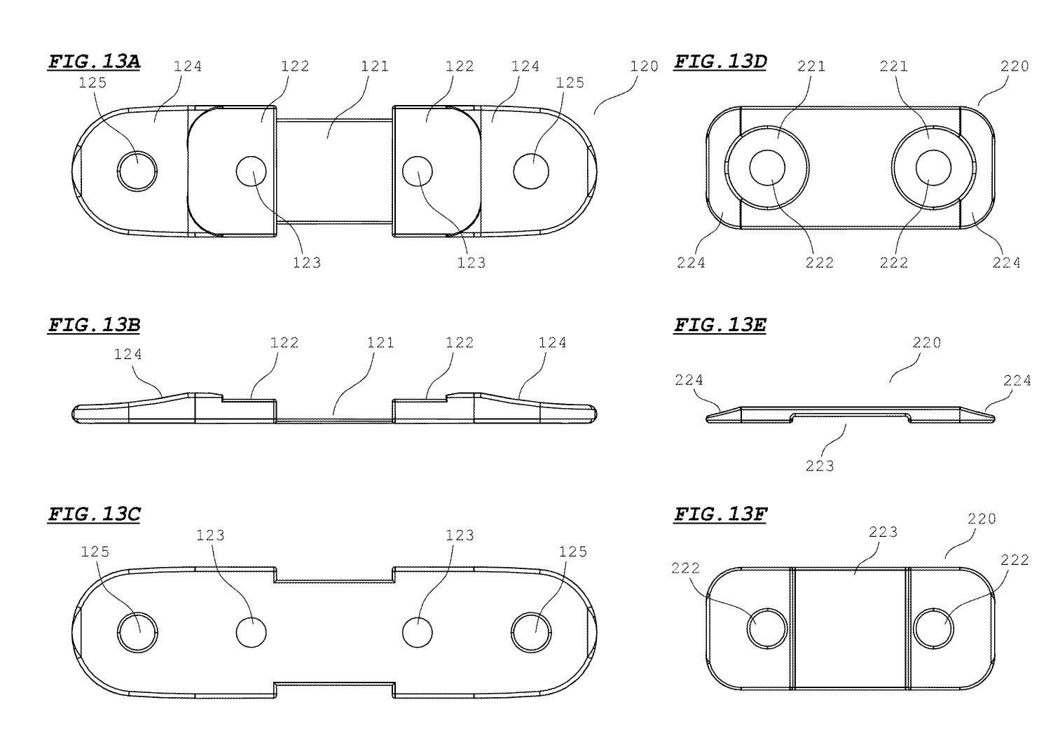

FIGS. 13A to 13C are a plan view, a side view, and a rear view, respectively, each showing a configuration of the base member 120 according to Embodiment 3.

With reference to FIG. 12 and FIGS. 13A to 13C, in a plan view, the base member 120 has a track-shaped contour, i.e., a contour obtained by connecting both ends of two parallel straight lines with arcs. The base member 120 has a shape that is symmetric with respect to the longitudinal direction and that is symmetric with respect to the width direction. The base member 120 has a length that allows the base member 120 to extend across a plurality of ribs.

The base member 120 includes a groove portion 121 extending in a direction orthogonal to the longitudinal direction. In the direction orthogonal to the longitudinal direction, the width of the bottom face of the groove portion 121 is smaller than the width of each part where a screw hole 123 of the base member 120 is formed. The part of the groove portion 121 has a thin plate shape, and thus, in this part, the base member 120 can be deflected in a direction perpendicular to the surface thereof. In a direction parallel to the longitudinal direction, the width of the groove portion 121 is slightly greater than the width of the bar attached thereto. The depth of the groove portion 121 is smaller than the thickness of the bar attached thereto. Therefore, when the bar is fitted into the groove portion 121, the upper face of the bar is at a position higher than the bottom faces of recessed portions 122.

The recessed portions 122 are formed at positions, on the upper face of the base member 120, that sandwich the groove portion 121. Further, the screw holes 123 are formed in the recessed portions 122, respectively. The recessed portions 122 have a shape that allows the pressing member 220 to be fitted therein. The screw holes 123 are formed at positions corresponding to holes 222 of the pressing member 220.

An inclined surface 124 is formed at an outer position, with respect to each recessed portion 122, on the upper face of the base member 120. A hole 125 is formed substantially at the center of each inclined surface 124. Each of the two inclined surfaces 124 is inclined so as to be increasingly close to the lower face of the base member 120, toward a corresponding end in the up-down direction. The two holes 125 are for allowing the base member 120 to be moved by a jig during surgery.

FIGS. 13D to 13F are a plan view, a side view, and a rear view, respectively, each showing a configuration of the pressing member 220 according to Embodiment 3.

With reference to FIG. 12 and FIGS. 13D to 13F, in a plan view, the pressing member 220 has a contour obtained by rounding corners of a rectangle. The pressing member 220 has a shape that is symmetric with respect to the longitudinal direction and that is symmetric with respect to the width direction. Two circular recessed portions 221 arranged in the longitudinal direction are formed on the upper face of the pressing member 220. A hole 222 for allowing the screw 320 to pass therethrough is formed at the center of each recessed portion 221. The diameter of the recessed portion 221 is slightly greater than the diameter of a head 320b of the screw 320. On the rear face of the pressing member 220, a groove portion 223 is formed at a position opposed the groove portion 121 of the base member 120.

The screw 320 includes a screw portion 320a and an umbrella-shaped head 320b. Different from Embodiments 1, 2 above, the screw 320 is not provided with a stopper.

The pressing member 220 is attached to the base member 120 by means of the screws 320 in a state where the pressing member 220 is fitted to the recessed portions 122 on the upper face of the base member 120. The screws 320 are screwed via the holes 222 into the screw holes 123 of the base member 120.

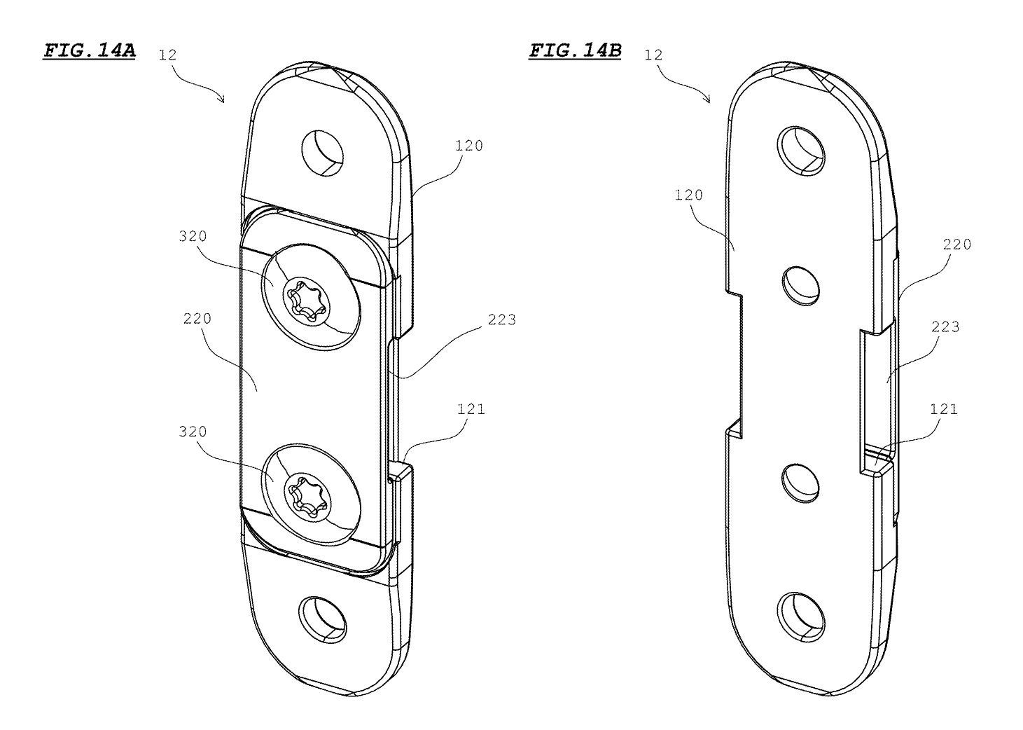

FIGS. 14A, 14B are perspective views showing a state where the fixture 12 according to Embodiment 3 has been assembled, viewed from the front side and the rear side, respectively.

In the case of the fixture 12 of Embodiment 3, in a state where the pressing member 220 is attached to the base member 120, the groove portion 121 on the base member 120 side and the groove portion 223 on the pressing member 220 side completely face each other. An end portion of a bar is sandwiched in the gap between the bottom face of the groove portion 121 and the bottom face of the groove portion 223. In a state where the screws 320 are completely fastened, the distance between the bottom face of the groove portion 121 and the bottom face of the groove portion 223 is smaller than the thickness of the bar. Therefore, when the screws 320 are fastened in a state where the end portion of the bar is sandwiched in the gap between the bottom face of the groove portion 121 and the bottom face of the groove portion 223, the upper face of the end portion of the bar is pressed by the bottom face of the groove portion 223 of the pressing member 220, and the lower face of the end portion of the bar is pressed against the bottom face of the groove portion 121 on the base member 120 side. As a result, the end portion of the bar is fixed to the fixture 12.

During surgery, the end portion of the bar is fitted into the groove portion 121 of the base member 120 in a state where the pressing member 220 is not attached to the base member 120. Here, the base member 120 is located at a position where the base member 120 extends across a plurality of ribs and where the base member 120 is most stabilized on the muscle layer. In this state, the pressing member 220 is fitted into the recessed portions 122 on the upper face of the base member 120, and further, the two screws 320 are screwed into the screw holes 123, respectively. When the two screws 320 are fastened, the end portion of the bar is pressed against the groove portion 121, whereby the end portion of the bar is fixed to the fixture 12, as described above. In Embodiment 3, the same operations are performed for the end portions of all the bars, whereby the fixture 12 is attached to each end portion.

<Effects of Embodiment 3>

Also in the case of the fixture 12 according to Embodiment 3, similar to Embodiments 1, 2 above, the end portion of the bar can be fixed to the fixture 12 through simple operations such as fitting the end portion of the bar into the groove portion 121, setting the base member 120 to the outside of a plurality of ribs so as to extend across the ribs, and fastening the screws 320. In addition, the end portion of the bar is slidable with respect to the groove portion 121. Therefore, in a state where the end portion of the bar is fitted in the groove portion 121, the fixture 12 can be located at a position on the muscle layer where the fixture 12 is most stabilized. If the screws 320 are fastened in this state, the end portion of the bar can be fixed to the fixture 12 at this position. Therefore, according to Embodiment 3, the bar can be stably secured to the chest of the patient through simple operations.

Since the pressing member 220 covers the groove portion 121, a large region of the upper face of the end portion of bar can be pressed by the pressing member. Accordingly, the end portion of the bar can be more rigidly fixed.

<Modification>

Embodiments of the present invention have been described. However, the present invention is not limited to the embodiments above, and various modifications can be made to the embodiments of the present invention.

For example, the shapes and contours of the fixtures shown in Embodiments 1 to 3, and the shapes and contours of the members forming each fixture are merely examples, and can be changed to other shapes and contours as appropriate.

In Embodiments 1, 2, only one side in the width direction of a bar is pressed by being fastened with a screw, and the other side thereof is caused to be engaged with a flange portion. However, the fixture may be configured such that both sides in the width direction of the bar are pressed by being fastened with screws.

In Embodiments 1, 2, each screw is provided with the stopper. However, a screw without a stopper as in Embodiment 3 may be used. However, in this case, an operation of fitting the screw to a screw hole is required during surgery, and thus, the operation during surgery becomes a little complicated when compared with Embodiments 1, 2.

In Embodiment 3, the pressing member 220 is configured to be removable from the base member 120. However, similar to Embodiment 1, the pressing member 220 may be supported by the base member 120 by means of a pin, such that the pressing member 220 is prevented from coming off from the base member 120 and is allowed to be close to and separated from the groove portion 121. The support means may not necessarily be a pin, and another support means may be used.

As the fixing screw, any screw having any structure can be used as long as the screw can attain fastening. A preferred screw is the one that is easy to attach and less likely to be loosened after having been attached. To this end, it is also preferable to provide a set of a screw and a dedicated screw-driving tool. Screws of various designs for loosening prevention have been proposed (for example, Japanese Patent No. 5249087, Japanese Patent No. 5336644, etc.). Such a screw may be used.

In Embodiments 1, 2, two bars can be fixed, but the number of bars that can be fixed is not limited thereto. For example, in the configuration shown in FIGS. 6A, 6B, the lengths of the support portion 105 and the long hole 106 may be further increased, such that a combination of the fixing member 200, the second pressing member 500, and the receiving member 300 can be further attached to the support portion 105.

Further, in Embodiments 1, 2, the receiving member is provided with the wall portions. However, the wall portions may be omitted. In Embodiment 2, the lower faces of the heads 411b, 412b of the screws 411, 412 are formed as flat surfaces. However, a means that could prevent the bars 21, 22 from slipping with respect to the lower faces of the heads 411b, 412b may be provided by, for example, providing protrusions, bosses, or the like to the lower faces of the heads 411b, 412b, or embossing the lower faces of the heads 411b, 412b. This also applies to the cases where the pressing member is used as in Embodiments 1, 3.

The projecting dimensions of the flange portions 404, 504 in Embodiment 1 can be adjusted as appropriate to dimensions that allow the upper faces of the bars 21, 22 to be appropriately pressed. The direction of the groove portion may not necessarily be orthogonal to the longitudinal direction of the base member, and may be any direction that crosses the longitudinal direction of the base member and that allows the base member to extend across a plurality of ribs.

As a precaution for a case where a star-shaped groove to be engaged with a screwdriver is damaged, it is preferable to separately provide the head of each screw with a means that allows rotation of the screw by means of another jig. For example, a plurality of dents may be provided to the surface of the head of the screw so as to be arranged in the circumferential direction thereof, so that projections of a jig can be fitted into the dents to turn the screw.

<Embodiment 4>

In Embodiments 1, 2 above, the fixing members 200, 210 are configured to be slidable, so that the fixing positions of the two bars 21, 22 can be adjusted. In Embodiment 4, a configuration different from those of Embodiments 1, 2 allows the fixing positions of the two bars 21, 22 to be easily adjusted.

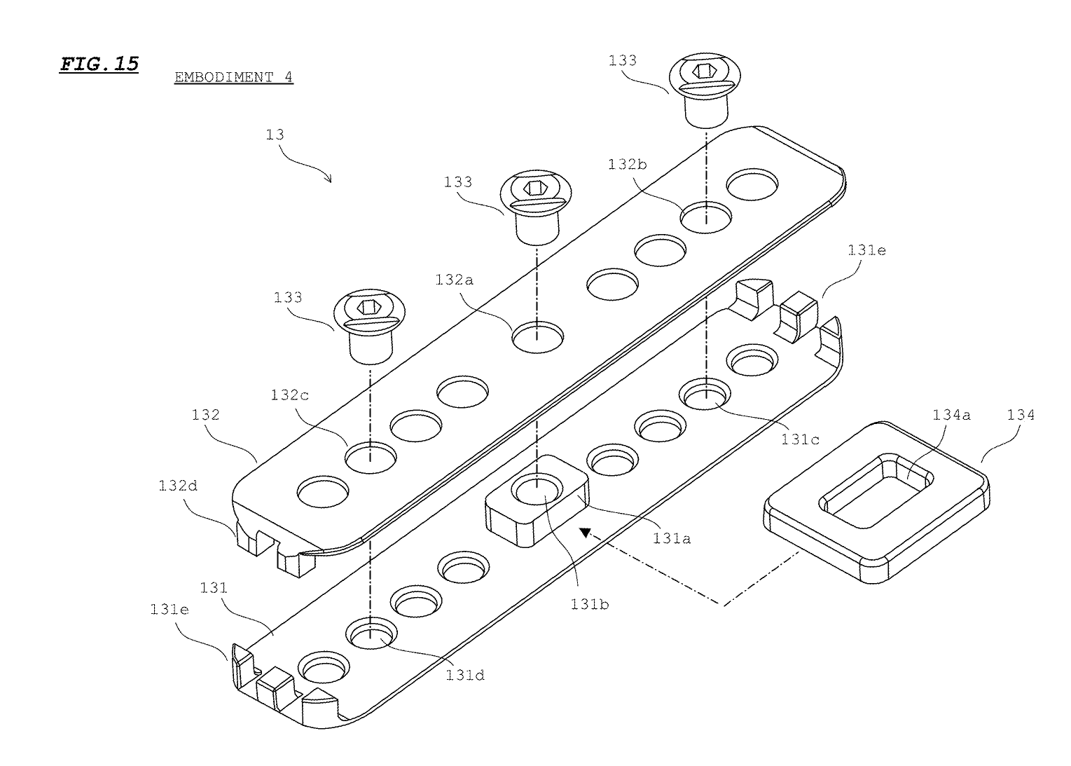

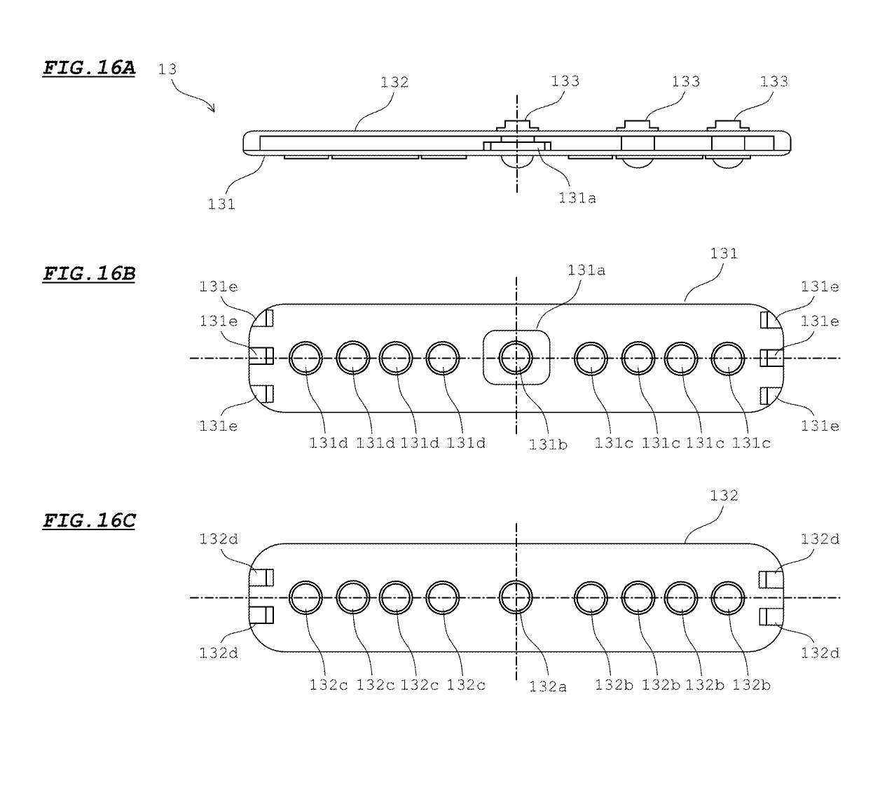

FIG. 15 is an exploded perspective view of a fixture 13 according to Embodiment 4. FIG. 16A is a side view showing a state where members according to Embodiment 4 have been assembled. FIG. 16B is a plan view of a base member according to Embodiment 4. FIG. 16C is a rear view of a pressing member according to Embodiment 4.

The fixture 13 includes a base member 131, a pressing member 132, screws 133, and a spacer 134. Similar to Embodiments 1 to 3 above, each member is formed from a material, such as titanium, that has good affinity to human bodies and that can ensure rigidity.

The base member 131 is formed as a plate-like member having, in a plan view, a contour obtained by rounding corners of a rectangle. The base member 131 has a shape symmetric with respect to the longitudinal direction and the short direction thereof. A protrusion 131a is provided at the center on the upper face of the base member 131. In a plan view, the protrusion 131a has a shape obtained by rounding corners of a rectangle. A screw hole 131b penetrating the protrusion 131a in the up-down direction is formed at the center of the protrusion 131a.

Four screw holes 131c penetrating the base member 131 in the up-down direction are provided on one side in the longitudinal direction of the base member 131 with respect to the protrusion 131a. Four screw holes 131d penetrating the base member 131 in the up-down direction are provided also on the other side in the longitudinal direction of the base member 131 with respect to the protrusion 131a. All the pitches between the four screw holes 131c and the pitches between the four screw holes 131d are the same. The screw hole 131b, the four screw holes 131c, and the four screw holes 131d are arranged side by side in a line in the longitudinal direction of the base member 131. In the lower face of the base member 131, the parts around the positions where the screw holes 131c and the screw holes 131d are provided protrude slightly downwardly. Accordingly, the base member 131 is thick at the positions where the screw holes 131c and the screw holes 131d are provided.

Three claw portions 131e upwardly protruding are provided to each end portion in the longitudinal direction of the base member 131. The three claw portions 131e provided to each end portion are arranged side by side in the short direction of the base member 131.

The pressing member 132 is formed as a plate-like member having, in a plan view, a contour obtained by rounding corners of a rectangle. The pressing member 132 has a shape symmetric with respect to the longitudinal direction and the short direction thereof. In the pressing member 132, a hole 132a penetrating the pressing member 132 in the up-down direction is provided at a position corresponding to the screw hole 131b on the base member 131 side. In addition, in the pressing member 132, holes 132b penetrating the pressing member 132 in the up-down direction are provided at positions corresponding to the four screw holes 131c on the base member 131 side. Further, holes 132c penetrating the pressing member 132 in the up-down direction are provided at positions corresponding to the four screw holes 131d on the base member 131 side.

Two claw portions 132d protruding downwardly are provided to each end portion in the longitudinal direction of the pressing member 132. The two claw portions 132d provided to each end portion are arranged side by side in the short direction of the pressing member 132. The two claw portions 132d are disposed at positions where the two claw portions 132d are fitted, without any substantial gap, between the three claw portions 131e on the base member 131 side. The protruding amount of each claw portion 132d is the same as the protruding amount of each claw portion 131e of the base member 131.

The spacer 134 has, in a plan view, a contour obtained by rounding corners of a rectangle. A hole 134a is provided at the center of the spacer 134. The hole 134a has a shape that allows the protrusion 131a of the base member 131 to be fitted therein without any substantial gap.

The thickness in the up-down direction of the spacer 134 is the same as the protruding amount of each claw portion 131e of the base member 131, and is slightly smaller than the thickness of the bar 21, 22. The protruding amount of the protrusion 131a of the base member 131 is the same as the thickness of the spacer 134.

When the fixture 13 is to be attached to end portions of the bars 21, 22, first, the spacer 134 is attached to the base member 131 such that the protrusion 131a of the base member 131 is fitted into the hole 134a. Next, the end portions of the two bars 21, 22 are disposed between the spacer 134 and the claw portions 131e on both sides. In this state, the pressing member 132 is overlaid on the base member 131 such that the claw portions 132d are fitted between the claw portions 131e, and a screw 133 is screwed into the screw hole 131b. Accordingly, the fixture 13 is attached in a temporary manner to the end portions of the bars 21, 22.

Then, a screw 133 is screwed into a screw hole 131c via a hole 132b. At this time, the screw 133 is inserted and screwed into a hole 132b, among the four holes 132b, that is at a position where the bar 21 can be sandwiched, without any substantial gap, between the screw 133 and a lateral face of the spacer 134. In addition, a screw 133 is screwed into a screw hole 131d via a hole 132c. Also at this time, the screw 133 is inserted and screwed into a hole 132c, among the four holes 132c, that is at a position where the bar 22 can be sandwiched, without any substantial gap, between the screw 133 and a lateral face of the spacer 134.

Since the two screws 133 are screwed in this manner, the end portions of the bars 21, 22 are supported by the fixture 13, without being dislocated in the longitudinal direction of the base member 131. Then, the screwed three screws 133 are further fastened. Accordingly, the pressing member 132 is pressed against the upper faces of the bars 21, 22. As a result, the fixture 13 is fixed to the end portions of the bars 21, 22.

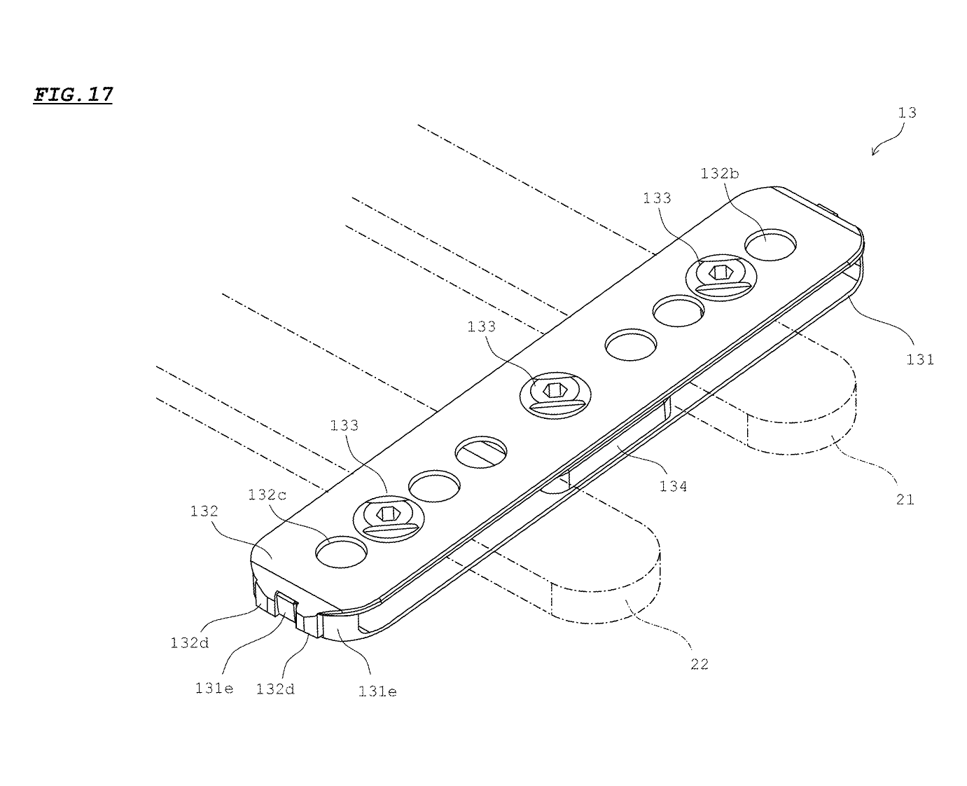

FIG. 17 shows a state where the fixture 13 is attached to the end portions of the bars 21, 22. As shown in FIG. 17, the bars 21, 22 are supported by the fixture 13 with an interval defined by the spacer 134. In a case where the interval between the bars 21, 22 is adjusted, another spacer is used.

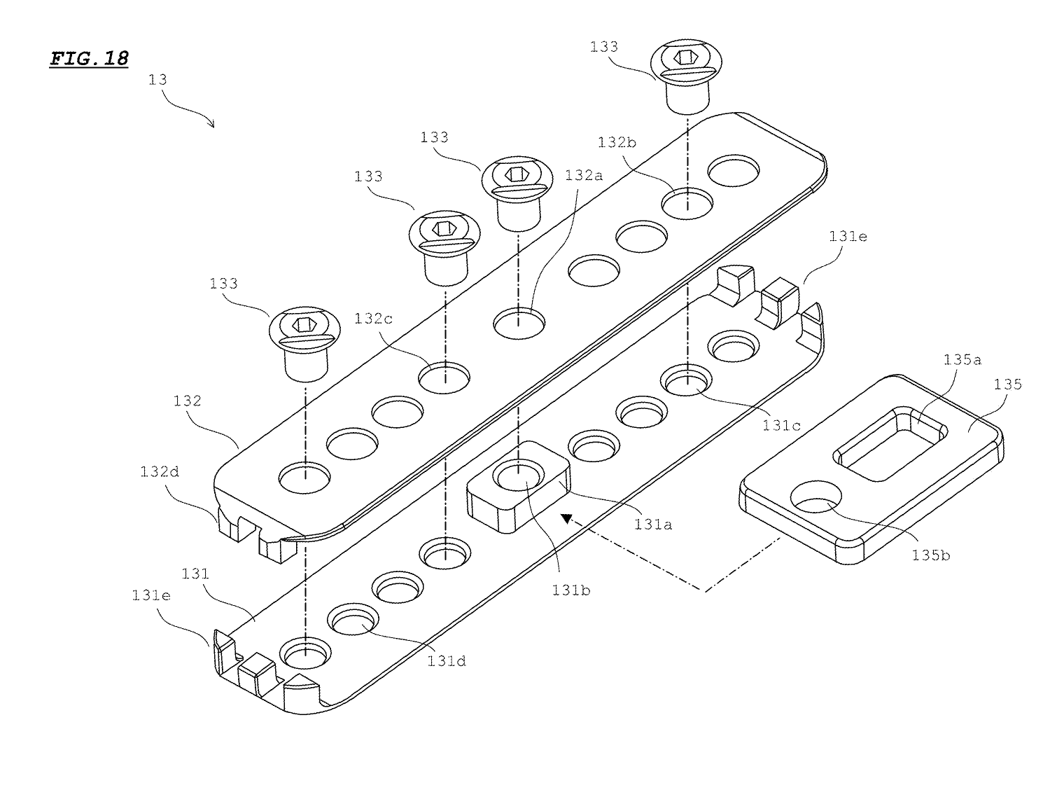

FIG. 18 is an exploded perspective view of a fixture when another spacer 135 is used.

The configurations of the base member 131 and the pressing member 132 are the same as those shown in FIG. 15. Here, the configuration of the spacer 135 is different from that shown in FIG. 15. The spacer 135 is longer in the longitudinal direction than the spacer 134 shown in FIG. 15. Specifically, one side in the longitudinal direction with respect to a hole 135a is extended in the longitudinal direction, and a hole 135b is provided in the extended region. The hole 135b is provided at a position corresponding to the screw hole 131d that is closest to the protrusion 131a among the four screw holes 131d formed in the base member 131. The hole 135a of the spacer 135 is equivalent to the hole 134a of the spacer 134 shown in FIG. 15.

Also when this spacer 135 is used, the fixture 13 is attached to the end portions of the bars 21, 22 in accordance with a procedure similar to that described above. However, in this case, a screw 133 that is to be passed through the hole 135b of the spacer 134 is added. This screw 133 is passed through the hole 135b of the spacer 134 via the hole 132c that is closest to the hole 132a, and is screwed into the screw hole 131d that is closest to the protrusion 131a. Another screw 133 that is screwed into a screw hole 131d is passed through the hole 132c that is farthest from the hole 132a, and is passed through the screw hole 131d that is farthest from the protrusion 131a.

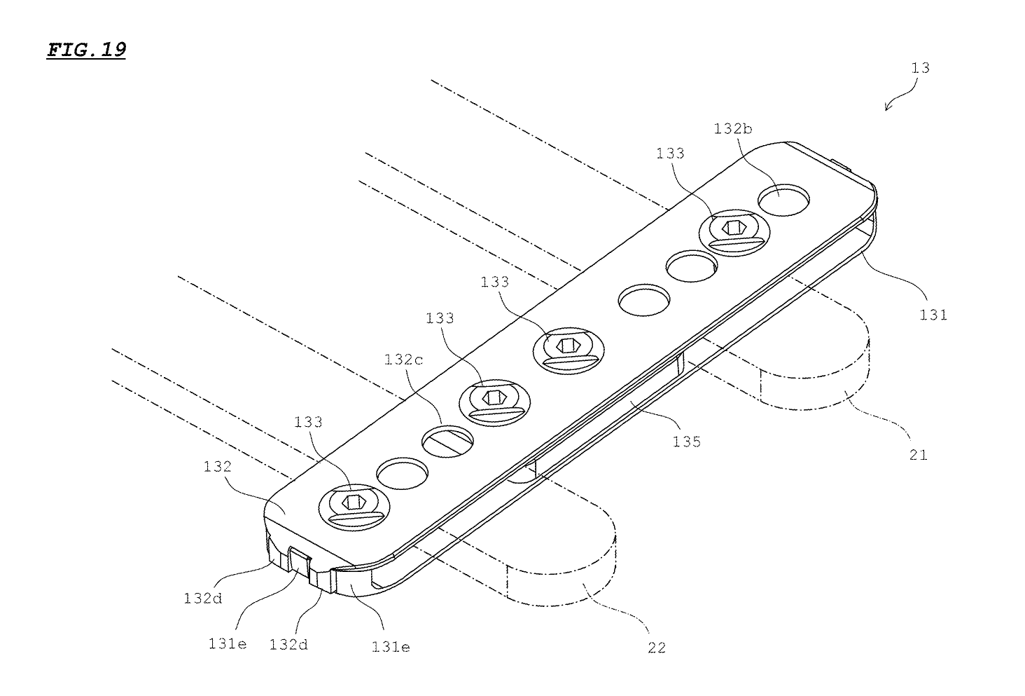

FIG. 19 shows a state where the fixture 13 is attached to the end portions of the bars 21, 22 with use of another spacer 135. In this case, the bar 21 is fixed to the fixture 13 at such a position as shown in FIG. 17. Meanwhile, the bar 22 is fixed to the fixture 13, at a position shifted in a direction toward an end portion of the base member 131, in a state of being sandwiched by the spacer 135 and the screw 133 screwed on the end portion side. Accordingly, the interval between the two bars 21, 22 is greater than that in the case of FIG. 17.

Thus, according to the configuration of Embodiment 4, simply by replacing the spacer 134 with the spacer 135, the fixing positions of the two bars 21, 22 can be easily adjusted. That is, if the spacer 135 that is suitable for the interval between two bars is used as appropriate, the bars can be sandwiched by the spacer 135 and the screws 133.

In the configuration shown in FIG. 18, the spacer 135 may be attached to the base member 131, with the spacer 135 rotated by 180 degrees, such that the hole 135b is located on the screw hole 131c side.

In the configuration example shown in FIG. 18, the spacer 135 is formed such that only one side thereof in the longitudinal direction with respect to the hole 135a is extended in the longitudinal direction. However, the spacer may be formed such that both of one side and the other side in the longitudinal direction with respect to the hole 135a may be extended in the longitudinal direction. If a spacer having such a configuration is used, the fixing positions of the two bars 21, 22 can be further separated from each other.

Furthermore, it is also possible to employ a method in which the two bars 21, 22 are fixed without the spacer 135 provided. In this case, the two bars 21, 22 are sandwiched by two screws 133 and lateral faces on both sides in the longitudinal direction of the protrusion 131a of the base member 131.

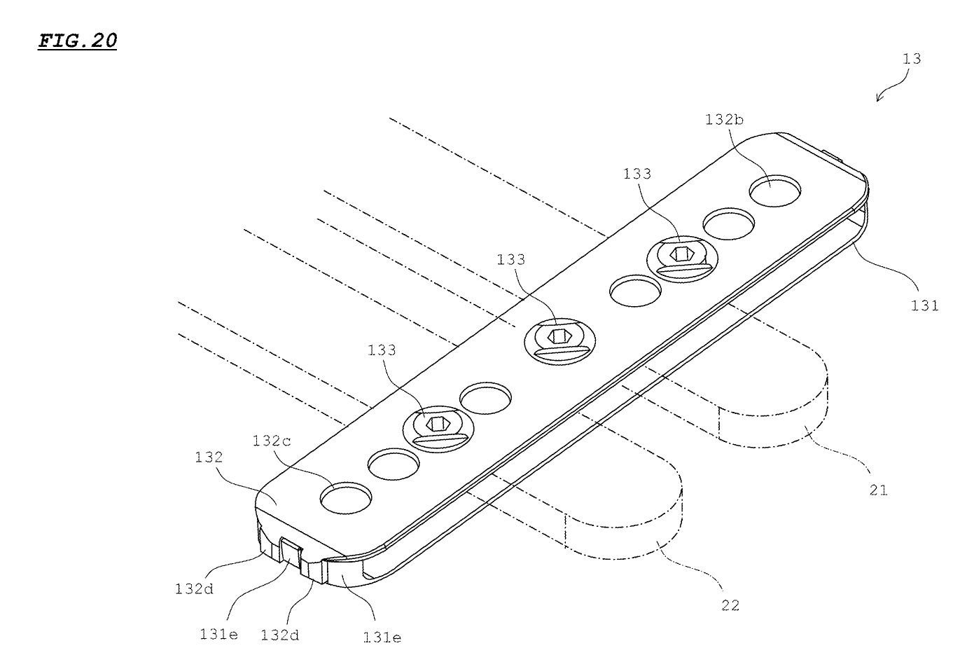

FIG. 20 shows a state where the fixture 13 is attached to the end portions of the bars 21, 22 without using a spacer. The interval between the two bars 21, 22 is smaller than that in the case of FIG. 17.

It should be noted that the screw holes 131c, 131d provided in the base member 131, and the holes 132b, 132c provided in the pressing member 132 are located at positions that allow the lateral faces of the screws 133 to be opposed, without any substantial gap, to the lateral faces of the bars 21, 22 attached to the fixture 13, for each of a case where various spacers are used and a case where no spacer is used, as described above. The spacer is also adjusted into a shape that allows the bars 21, 22 to be sandwiched, without any substantial gap, by the screws 133 and the lateral faces of the spacer.

Since the base member 131 and the pressing member 132 are provided with a plurality of screw holes 131c, 131d and a plurality of holes 132b, 132c arranged side by side in the longitudinal direction, the fixing operation in accordance with the interval between the bars 21, 22 can be easily performed, simply by changing the screwing positions as appropriate. That is, if the screw holes 131c, 131d for screwing are changed, the interval between the fixing positions of the two bars 21, 22 can be adjusted. Therefore, through simple operations, the two bars 21, 22 can be smoothly fixed to optimum positions for the state of ribs 32 of the patient.

In addition to the above, various modifications can be made as appropriate to the embodiments of the present invention, without departing from the scope of the technological idea defined by the claims.

What is claimed is:

1. A fixture for securing a bar for funnel chest correction to a chest of a patient, the fixture comprising:
a base member having a length that allows the base member to extend across a plurality of ribs;
a pressing member configured to be overlaid on the base member;
a protrusion formed on an upper face of the base member;
a plurality of screw holes formed in the base member so as to be arranged side by side in a longitudinal direction on both sides of the protrusion of the base member;
a plurality of holes formed in the pressing member at positions corresponding to the respective screw holes; and
a plurality of screws, wherein
the pressing member is overlaid on the base member in a state where two bars are respectively disposed on both sides of the protrusion, and a screw is screwed into any of the screw holes on each of both sides of the protrusion, whereby the two bars are fixed by being sandwiched by the base member and the pressing member.

2. The fixture for the bar for funnel chest correction according to claim 1, further comprising
a spacer configured to be fitted to the protrusion, wherein
when the two bars are sandwiched by the base member and the pressing member and fixed by the screws in a state where the spacer is fitted to the protrusion, the two bars are sandwiched in a width direction by the screws and lateral faces on both sides in the longitudinal direction of the spacer.

3. A fixture for securing a bar for funnel chest correction to a chest of a patient, the fixture comprising:
a base member having a length that allows the base member to extend across a plurality of ribs;
a groove portion that is formed in the base member and into which an end portion of the bar is slidably fitted;
a screw hole formed in a face on the groove portion side of the base member;
a screw configured to be screwed into the screw hole; and
a pressing part configured to press the end portion of the bar fitted in the groove portion, against the groove portion through fastening of the screw onto the base member, thereby fixing the end portion to the groove portion.

4. The fixture for the bar for funnel chest correction according to claim 3, wherein
the screw hole is provided at a position separated by a predetermined distance from the groove portion,
the screw is configured such that a head thereof extends above the groove portion when the screw is screwed into the screw hole,
the pressing part includes the head of the screw, and
when the screw is fastened into the screw hole in a state where the end portion of the bar is fitted in the groove portion, a lower face of the head of the screw presses an upper face of the bar, and the end portion of the bar is pressed against the groove portion.

5. The fixture for the bar for funnel chest correction according to claim 4, wherein
a recessed portion to which the head of the screw is fitted and which is continuous with the groove portion is formed around the screw hole of the base member.

6. The fixture for the bar for funnel chest correction according to claim 4, wherein
the screw hole is provided only on one side in a longitudinal direction of the base member with respect to the groove portion,
a flange portion projecting inwardly toward the groove portion from a boundary on another side in the longitudinal direction of the groove portion is provided to the base member, and
while a part on the other side of the end portion of the bar is prevented from coming off by the flange portion, the upper face on the one side of the end portion of the bar is pressed by the head of the screw, whereby the end portion is fixed to the groove portion.

7. The fixture for the bar for funnel chest correction according to claim 3, wherein
the pressing part includes a pressing member configured to be fixed to the base member by means of the screw, and
when the screw is fastened into the screw hole in a state where the end portion of the bar is fitted in the groove portion, a lower face of the pressing member presses an upper face of the bar and the end portion of the bar is pressed against the groove portion.

8. The fixture for the bar for funnel chest correction according to claim 4, wherein
the screw hole is provided only on one side in a longitudinal direction of the base member with respect to the groove portion,
a flange portion projecting inwardly toward the groove portion from a boundary on another side in the longitudinal direction of the groove portion is provided to the base member, and
while a part on the other side of the end portion of the bar is prevented from coming off by the flange portion, the upper face on the one side of the end portion of the bar is pressed by the pressing member, whereby the end portion is fixed to the groove portion.

9. The fixture for the bar for funnel chest correction according to claim 7, comprising
a support member that allows the pressing member to be supported by the base member such that the pressing member is prevented from coming off from the base member and is allowed to be close to and separated from the groove portion.

10. The fixture for the bar for funnel chest correction according to claim 7, wherein
the screw hole is provided on each side in a longitudinal direction of the base member with respect to the groove portion,
the pressing member has a size that covers the groove portion, and
the pressing member is overlaid on the base member so as to cover the groove portion in a state where the end portion of the bar is fitted in the groove portion, and the screw is fastened in each of the two screw holes, whereby the lower face of the pressing member presses the upper face of the bar and the end portion of the bar is pressed against the groove portion.

11. The fixture for the bar for funnel chest correction according to claim 3, wherein
the base member includes a support portion extending, in a direction in a longitudinal direction of the base member, away from a position of the groove portion, and the support portion has a long hole extending in the longitudinal direction formed therein,
the fixture comprises:
a fixing member to which an end portion of another bar is attached;
another groove portion that is formed in the fixing member and into which the end portion of the other bar is slidably fitted;
a receiving member that is disposed on a side opposite to the fixing member with respect to the support portion and to which the fixing member is screwed by another screw via the long hole; and
another pressing part configured to press the end portion of the other bar fitted in the other groove portion, against the other groove portion through fastening of the other screw onto the receiving member, thereby fixing the end portion of the other bar to the other groove portion.

12. The fixture for the bar for funnel chest correction according to claim 11, wherein
a width, of the fixing member, in a direction orthogonal to the longitudinal direction is substantially identical to a width, of the support portion, in a direction orthogonal to the longitudinal direction, and
the receiving member includes a pair of wall portions which sandwich, in a direction orthogonal to the longitudinal direction, the fixing member overlaid on the support portion, together with the support portion.

13. The fixture for the bar for funnel chest correction according to claim 11, wherein
the fixing member includes a cut groove that allows the other screw to pass in the longitudinal direction.

14. A funnel chest correction device comprising:
a fixture configured to secure a bar for funnel chest correction to a chest of a patient; and
the bar for funnel chest correction of which end portion is to be fixed to the fixture, wherein
the fixture includes:
a base member having a length that allows the base member to extend across a plurality of ribs;
a pressing member configured to be overlaid on the base member;
a protrusion formed on an upper face of the base member;
a plurality of screw holes formed in the base member so as to be arranged side by side in a longitudinal direction on both sides of the protrusion of the base member;
a plurality of holes formed in the pressing member at positions corresponding to the respective screw holes; and
a plurality of screws, and
the pressing member is overlaid on the base member in a state where two bars are respectively disposed on both sides of the protrusion, and a screw is screwed into any of the screw holes on each of both sides of the protrusion, whereby the two bars are fixed by being sandwiched by the base member and the pressing member.

15. A funnel chest correction device comprising:
a fixture configured to secure a bar for funnel chest correction to a chest of a patient; and
the bar for funnel chest correction of which end portion is to be fixed to the fixture, wherein
the fixture includes:
a base member having a length that allows the base member to extend across a plurality of ribs;
a groove portion that is formed in the base member and into which an end portion of the bar is slidably fitted;
a screw hole formed in a face on the groove portion side of the base member;
a screw configured to be screwed into the screw hole; and
a pressing part configured to press the end portion of the bar fitted in the groove portion, against the groove portion through fastening of the screw onto the base member, thereby fixing the end portion to the groove portion.
